(19) United States
(12) Patent Application Publication
(10) Pub. No.: US 2026/0130928 A1
(43) Pub. Date: May 14, 2026

(54) COMBINATION OF AKR1C3 ACTIVATED ANTICANCER PRODRUG COMPOUND AND ANALGESIC DRUG FOR TREATING CANCER PATIENTS ACCOMPANIED BY PAIN

(71) Applicant: ASCENTAWITS PHARMACEUTICALS, LTD., Pingshan District, Shenzhen, Guangdong (CN)

(72) Inventors: Jianxin DUAN, Shenzhen, Guangdong (CN); Yanbin XIE, Shenzhen, Guangdong (CN); Fanying MENG, San Francisco, CA (US); Jing HAO, Shenzhen, Guangdong (CN); Tianyang QI, Shenzhen, Guangdong (CN); Xing LIU, Shenzhen, Guangdong (CN); Bing LI, Shenzhen, Guangdong (CN)

(21) Appl. No.: 19/120,073

(22) PCT Filed: Oct. 12, 2023
(86) PCT No.: PCT/CN2023/124204

§ 371 Date: Apr. 10, 2025

(30) Foreign Application Priority Data
Oct. 12, 2022 (CN) 202211246436.X

(51) Int. Cl. A61K 31/675; A61K 31/167; A61K 31/192; A61K 31/196; A61K 31/235; A61K 31/365; A61K 31/405; A61K 31/407; A61K 31/60; A61K 31/616; A61K 31/635; A61P 29/00; A61P 35/00

(52) U.S. Cl. CPC A61K 31/675; A61K 31/167; A61K 31/192; A61K 31/196; A61K 31/235; A61K 31/365; A61K 31/405; A61K 31/407; A61K 31/60; A61K 31/616; A61K 31/635; A61P 29/00; A61P 35/00

(57) ABSTRACT

The present invention provides a method for treating cancer patients accompanied by pain by combining an AKR1C3-activated anticancer prodrug compound with an analgesic drug, a pharmaceutical use, a drug combination and a pharmaceutical formulation thereof, which provides a reasonable alternative for clinical cancer treatment. In particular, the combination of AST-3424 and non-steroidal analgesic drug can achieve the effect of simultaneously treating cancer and relieving pain.

### TECHNICAL FIELD

The invention relates to a method for treating tumors and cancers, and in particular to a method for treating cancers and tumors by combining an AKR1C3-activated anticancer prodrug compound with a non-steroidal analgesic drug, and belongs to the field of tumor treatment.

### BACKGROUND

AST-3424, a DNA alkylating agent prodrug targeting overexpressed aldehyde-keto reductase 1C3 (AKR1C3) (WO2016145092, WO2017087428), has a CAS number of 2097713-69-2 and has the following structure:

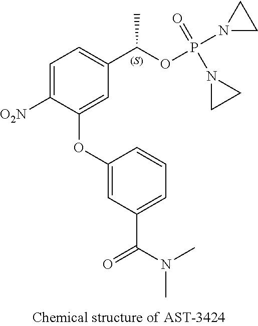

When entering into cancer cells, AST-3424 (also known as OBI-3424, TH-3424) is activated by the AKR1C3 enzyme overexpressed by cancer cells to release a metabolite AST-2660 (also known as AST-2660). AST-3424 itself has low toxicity to cancer cells. In its animal model and in vitro pharmacological experiments, the pharmacological effect of AST-3424 is associated with the expression of the AKR1C3 enzyme: the prodrug AST-3424 is metabolized by AKR1C3 enzyme in the presence of NADPH to AST-2660, and its potency is positively correlated with AKR1C3 expression levels (Meng F, Li W F, Jung D, et al. A novel selective AKR1C3-activated prodrug AST-3424/OBI-3424 exhibits broad anti-tumor activity. Am J Cancer Res. 2021; 11(7):3645-3659; Evans K, Duan J, Pritchard T, et al. OBI-3424, a Novel AKR1C3-Activated Prodrug, Exhibits Potent Efficacy against Preclinical Models of T-ALL. Clin Cancer Res. 2019; 25(14):4493-4503. doi:10.1158/1078-0432.CCR-19-0551; Wang Y, Liu Y, Zhou C, et al. An AKR1C3-specific prodrug with potent anti-tumor activities against T-ALL. Leuk Lymphoma. 2020; 61(7):1660-1668. doi:10.1080/10428194.2020.1728746; He P, Wang C, Wang Y, et al. A Novel AKR1C3 Specific Prodrug TH3424 With Potent Antitumor Activity in Liver Cancer [retracted in: Clin Pharmacol Ther. 2021 July; 110(1):262]. Clin Pharmacol Ther. 2021, 110(1):229-237. doi:10.1002/cpt.2171).

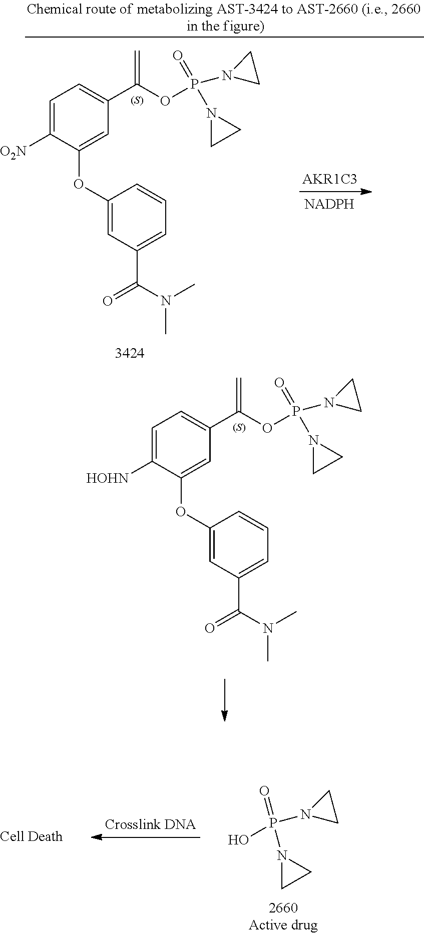

Currently, the drug has entered Phase II clinical trials in China and the United States (US NCT03592264, castration-resistant prostate cancer and liver cancer; US NCT04315324, T-cell acute lymphoblastic leukemia (T-ALL); China CTR20191399, various solid tumors; CTR20201915, T-cell acute lymphoblastic leukemia and B-cell acute lymphoblastic leukemia).

In particular, the results of the Phase I clinical trial in the United States also revealed that the therapeutic effect of AST-3424 is highly correlated with the expression of the AKR1C3 enzyme. Patients with high expression of the AKR1C3 enzyme will have a better therapeutic effect. It has been preliminarily determined that patients with H-score≥135 will be elected to enroll in the subsequent Phase II clinical trial (Tsimberidou, Apostolia & Verschraegen, Claire & Hsu, Pei & Pearce, Tillman. (2022). Safety, pharmacokinetics, and clinical activity of OBI-3424, an AKR1C3-activated prodrug, in patients with advanced or metastatic solid tumors: A phase 1 dose-escalation study. Journal of Clinical Oncology. 40. 3030-3030.10.1200/JCO.2022.40.16_suppl.3030).

### SUMMARY OF THE INVENTION

As the sponsor of the Phase I clinical trial in China, the applicant found in some advanced solid tumor cases reported in the clinical trial that: some patients had higher AKR1C3 enzyme expression and showed a better response to AST-3424, whereas some patients had almost the same higher AKR1C3 enzyme expression but showed almost no response to AST-3424. After investigation, it was found that some patients who had no response had been administered with non-steroidal analgesic drugs during the AST-3424 treatment course.

The literature studies have revealed that non-steroidal analgesic drugs have AKR1C3 inhibitory effects: Proc. Natl. Acad. Sci. USA 80 (1983), Inhibition of a major NAD(P)-linked oxidoreductase from rat liver cytosol by steroidal and nonsteroidal anti-inflammatory agents and by prostaglandins, reported that seven drugs, including Aspirin, Sodium salicylate, Paracetamol, Diclofenac, Fenoprofen, Flufenamic acid and Tolmetin, have AKR1C3 inhibitory activity;
Y. Higaki et al., Chemico-Biological Interactions, 143-144 (2003) 50-513, Selective and potent inhibitors of human 20a-hydroxysteroid dehydrogenase (AKR1C1) that metabolizes neurosteroids derived from progesterone, reported that Indomethacin has AKR1C3 inhibitory activity;
Biochem Pharmacol. 2008 Jan. 15; 75(2): 484-493. “An Indomethacin Analogue, N-(4-Chlorobenzoyl)-melatonin, is a Selective Inhibitor of Aldo-keto Reductase 1C3 (Type 2 3α-HSD, Type 5 17β-HSD, and Prostaglandin F Synthase), a Potential Target for the Treatment of Hormone Dependent and Hormone Independent Malignancies”, reported that six drugs, including Naproxen, Ibuprofen, Flurbiprofen, Zomepirac, Meclofenamic Acid and Sulindac, have AKR1C3 inhibitory activity;
Mol Pharmacol 67:60-68, 2005, “Development of Nonsteroidal Anti-Inflammatory Drug Analogs and Steroid Carboxylates Selective for Human Aldo-Keto Reductase Isoforms: Potential Antineoplastic Agents That Work Independently of Cyclooxygenase Isozymes”, reported that two drugs, including Celecoxib and Mefenamic acid, have AKR1C3 inhibitory activity.

However, it is uncertain whether the inhibition of such non-steroidal analgesic drugs on the activity of AKR1C3 would definitely affect the process of metabolizing the AKR1C3-activated anticancer prodrug compounds such as AST-3424 by AKR1C3 enzyme to the active substance AST2660, and further impact the efficacy of AST-3424.

In this regard, the applicant conducted in vivo and in vitro experiments, which proved that certain non-steroidal analgesic drugs would affect the efficacy of AST-3424 if used in combination with AST-3424, while other non-steroidal analgesic drugs would not.

Cancer patients often experience various pains due to various reasons. Clinically, cancer patients at any stage are likely to experience pain, especially in advanced cancer: data show that the proportion of clinically cured cancer patients accompanied by pain is about 34%, the proportion of newly diagnosed cancer patients accompanied by pain is about 25%, 30% to 50% of patients experience pain during treatment, and the proportion of advanced cancer patients accompanied by pain can reach 70-90%. Since pain seriously affects the quality of life of patients, most patients will be administered analgesic drugs under the guidance of medical professionals. Symptomatic intervention and treatment are carried out in accordance with the three-step analgesic ladder of the World Health Organization. In the first step, oral analgesic drugs are administered as the main method, and non-steroidal common analgesic drugs are used to relieve pain. In the second step, if the first-step regimen cannot relieve pain, it is necessary to change to weak opioid drugs, such as codeine, tramadol sustained-release tablets and other second-step drugs for cancer pain treatment. In the third step, if the above-mentioned step treatments cannot relieve pain, powerful opioid drugs such as oxycodone, morphine, fentanyl, etc. are needed.

Considering that cancer pain is very common, and that patients in clinical practice are often administered with non-steroidal analgesic drugs for intervention and symptomatic treatment of pain at the same time as being administered with cancer treatment drugs, the applicant provides the following technical solution based on the above clinical trial results and the experimental results that some non-steroidal analgesic drugs do not affect the efficacy of AST-3424.

A method for treating cancer patients accompanied by pain by combining a drug containing an AKR1C3-activated anticancer prodrug compound or its salt, ester, solvate or isotope isomer with a non-steroidal analgesic drug.

The pharmaceutical use of an AKR1C3-activated anticancer prodrug compound or its salt, ester, solvate or isotope isomer in the manufacture of a medicament for treating cancer patients accompanied by pain in combination with a non-steroidal analgesic drug.

The present invention also provides a drug combination, which comprises the following substances as active ingredients:
an AKR1C3-activated prodrug compound or its salt, ester, solvate or isotope isomer, and
a non-steroidal analgesic drug;
wherein, the active ingredients are formulated together or separately for combined use, simultaneous use or separate use.

The drug combination of the present invention is used for treating cancer or tumor patients accompanied by pain.

AKR1C3-activated anticancer prodrug compounds are compounds that are metabolized by AKR1C3 enzyme to DNA alkylating agents.

In general, AKR1C3-activated anticancer prodrug compounds meet, but are not limited to, at least one of the following conditions:

A. The inhibitory effect of a compound on cancer cell proliferation detected in the presence of an AKR1C3 inhibitor (for example, compound 36, i.e.

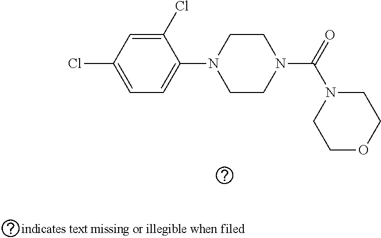 [SMILES: O=C(N1CCOCC1)N1CCN(c2ccc(Cl)cc2Cl)CC1]

in Bioorganic and Medicinal Chemistry, 2014: 962-977, also referred to as TH-3021 in the specification) is smaller than that detected in the absence of an AKR1C3 inhibitor. When the inhibitory effect on cancer cell proliferation is quantified with IC₅₀, if the IC₅₀ of a compound on a cancer cell line detected in the presence of an AKR1C3 inhibitor is greater than the IC₅₀ detected in the absence of an AKR1C3 inhibitor, it can be determined that the compound is an AKR1C3-activated anticancer drug (Lysis-Prodrug). Specific compounds are as the compounds disclosed in the following patent documents:
PCT/US2016/021581 with Publication No. WO2016145092A1, corresponding to Chinese Patent Application No. 2016800150788 with Publication No. CN107530556A;
PCT/US2016/062114 with Publication No. WO2017087428, corresponding to Chinese Patent Application No. 2016800446081 with Publication No. CN108290911A;
PCT/US2016/025665 with Publication No. WO2016161342, corresponding to Chinese Patent Application No. 2016800200132 with Publication No. CN108136214A; and
PCT/NZ2019/050030 with Publication No. WO2019190331, corresponding to Chinese Patent Application No. CN2019800234236 with Publication No. CN111918864A,
PCT/CN2020/120281 with Publication No. WO2021068952, corresponding to Chinese Patent Application No. CN2020800716528 with Publication No. CN114555574A.

The entire contents of above patent documents are incorporated herein by reference in its entirety.

The compounds disclosed in PCT/US2016/021581 with Publication No. WO2016145092A1 (corresponding to Chinese Patent Application No. 2016800150788 with Publication No. CN107530556A), PCT/US2016/062114 with Publication No. WO2017087428 (corresponding to Chinese Patent Application No. 2016800446081 with Publication No. CN108290911A) and PCT/US2016/025665 with Publication No. WO2016161342 (corresponding to Chinese Patent Application No. 2016800200132 with Publication No. CN108136214A) are lysis-prodrugs that are finally metabolized to primary drugs such as

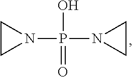

paclitaxel and camptothecin; the compounds disclosed in PCT/NZ2019/050030 with Publication No. WO2019190331 (corresponding to Chinese Patent Application No. CN2019800234236 with Publication No. CN111918864A) are lysis-prodrugs that are finally metabolized to nitrogen mustard drugs as primary drugs.

B. The inhibitory effect of a compound on the proliferation of cancer cells with different expression levels of AKR1C3 enzyme is significantly different, and the inhibitory effect on the proliferation of cancer cells with high expression of AKR1C3 enzyme is much greater than that of cancer cells with low expression of AKR1C3 enzyme. When the inhibitory effect on cancer cell proliferation is quantified with IC₅₀, if the IC₅₀ value of a compound on cancer cells with high expression of AKR1C3 enzyme is lower than the IC₅₀ value on cancer cells with low expression of AKR1C3 enzyme, it can be determined that the compound is an AKR1C3-activated anticancer drug. Specific compounds are as the compounds disclosed in Patent PCT/CN2020/120281 with Publication No. WO2021068952A1, and PCT/IB2020/057285 with Publication No. WO2021005586 (corresponding to Chinese Patent Application No. 2020800538041 with Publication No. CN114206870A). The entire contents of these patent documents are incorporated herein by reference in its entirety.

Regarding the drug described herein, it refers to a medicament or formulation. The prepared medicament contains a specific dosage range of active ingredient compounds or salts or solvates thereof, and/or the prepared formulation is in a specific dosage form and is administered via a specific mode of administration.

The prepared medicament, drug and formulation may further contain pharmaceutically acceptable adjuvants or excipients. The medicament can be in any dosage form for clinical administration, such as tablets, suppositories, dispersible tablets, enteric-coated tablets, chewable tablets, orally disintegrating tablets, capsules, sugar-coated agents, granules, dry powders, oral solutions, a small needle for injection, lyophilized powder for injection, or infusion solutions. According to the specific dosage form and the mode of administration, the pharmaceutically acceptable adjuvants or excipients in the medicament may include one or more of the following: diluent, solubilizer, disintegrant, suspension, lubricant, adhesive, filler, flavoring agent, sweetener, antioxidant, surfactant, preservative, wrapping agent and pigment.

Monotherapy refers to single drug therapy. Combination therapy refers to combined drug therapy. Single drug therapy refers to the use of only one anticancer drug in one course of treatment. Combination therapy refers to the use of two or more anticancer drugs simultaneously or successively in one course of treatment.

Generally speaking, for combination therapy, different administration dosages and administration cycles need to be explored according to the characteristics of the disease and the types of drugs to be used in combination; and only with the exploration according to the above conditions, the combined drug therapy regimen may achieve better therapeutic effect as compared with single drug therapy.

The administration dosage, administration cycle, and administration regimen of monotherapy and combination therapy regimens are all explored through clinical trials.

Regarding the compounds described herein, since their chemical structures contain an organic amine structure and a P═O double bond structure, the compounds may also be administered in the form of salts. In other words, the present invention provides pharmaceutically acceptable salts of the compounds shown, wherein the salts may be basic salts, including salts formed by the compounds and inorganic bases (such as alkali metal hydroxides or alkaline earth metal hydroxides) or organic bases (such as monoethanolamine, diethanolamine or triethanolamine). Alternatively, the salts may be acid salts, including salts formed by the compounds and inorganic acids (such as hydrochloric acid, hydrobromic acid, hydroiodic acid, nitric acid, perchloric acid, sulfuric acid or phosphoric acid) or organic acids (such as methanesulfonic acid, trifluoromethanesulfonic acid, ethanesulfonic acid, benzenesulfonic acid, p-toluenesulfonic acid, fumaric acid, oxalic acid, maleic acid or citric acid). Analogously, since the compounds may also react with certain acids or alcohols to form esters, they may also be administered in the form of esters.

Analogously, the above-mentioned compound concept also includes various crystal forms of the compounds. For example, PCT application PCT/CN2023/080261 with publication No. WO2023169462A1 discloses a crystal form of AKR1C3-activated anticancer prodrug compound:

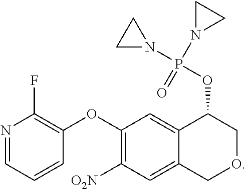 [SMILES: O=[N+]([O-])c1cc2c(cc1Oc1cccnc1F)[C@H](OP(=O)(N1CC1)N1CC1)COC2]

For various reasons, these compounds may also form solvates with certain solvents. The solvates are hydrates or alcoholates. Accordingly, the compounds may also be administered in the form of solvates. The selection and preparation of acceptable salts, esters and solvates of compounds are well known in the art.

The term “isotopic variant” refers to a compound containing non-natural proportions of isotopes at one or more of the atoms that constitute such a compound. In some embodiments, an “isotopic variant” of a compound contains non-natural proportions of one or more isotopes, including (but not limited to) hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²¹I), and iodine-131 (¹³¹I). In some embodiments, an “isotopic variant” of a compound is in a stable form (i.e., non-radioactive). In some embodiments, an “isotopic variants” of a compound contains non-natural proportions of one or more isotopes, including (but not limited to) hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In some embodiments, an “isotopic variant” of a compound is in an unstable form (i.e., radioactive). In some embodiments, an “isotopic variant” of a compound contains non-natural proportions of one or more isotopes, including (but not limited to) tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I) It should be understood that in the compounds as provided herein, when deemed feasible by those skilled in the art, any hydrogen may be for example ²H (i.e., D), or any carbon may be for example ¹³C, or any nitrogen may be for example ¹⁵N, and any oxygen may be ¹⁸O. In some embodiments, an “isotopic variant” of a compound contains non-natural proportions of deuterium (D).

Non-steroidal analgesic drugs are a large class of drugs, and because they often bring about anti-inflammatory effect, they are also called “non-steroidal anti-inflammatory analgesic drugs”. From the perspective of structure, non-steroidal antipyretic analgesic drugs refer to a class of antipyretic analgesics that do not contain steroidal structures in their chemical structure, wherein some varieties have anti-inflammatory and anti-rheumatic effects. The non-steroidal analgesic drugs used clinically include the following varieties:

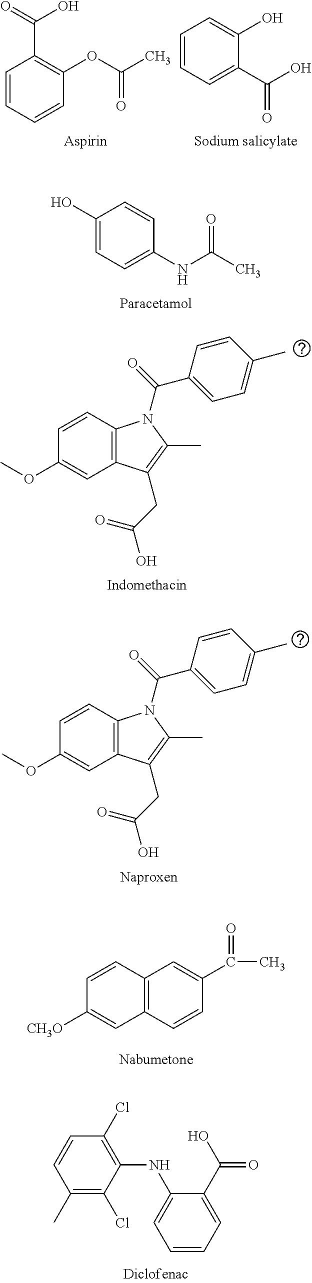

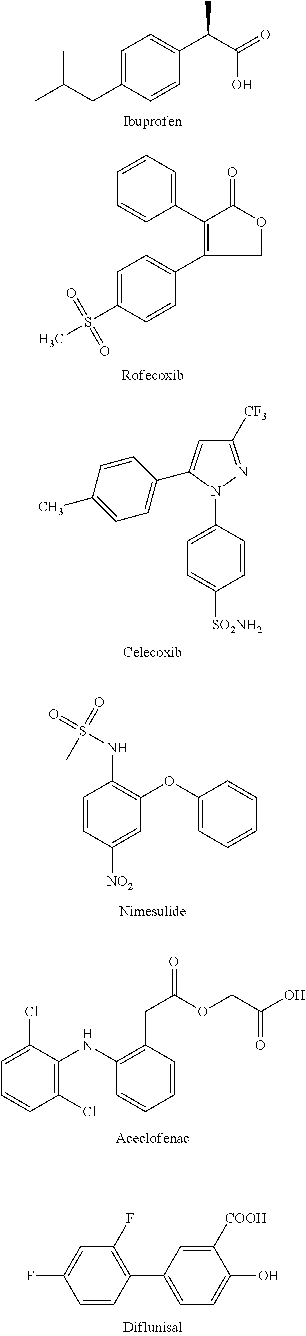 [SMILES: CC(C)Cc1ccc([C@@H](C)C(=O)O)cc1 ; CS(=O)(=O)c1ccc(C2=C(c3ccccc3)C(=O)OC2)cc1 ; Cc1ccc(-c2cc(C(F)(F)F)nn2-c2ccc(S(N)(=O)=O)cc2)cc1 ; CS(=O)(=O)Nc1ccc([N+](=O)[O-])cc1Oc1ccccc1 ; O=C(O)COC(=O)Cc1ccccc1Nc1c(Cl)cccc1Cl ; O=C(O)c1cc(-c2ccc(F)cc2F)ccc1O]

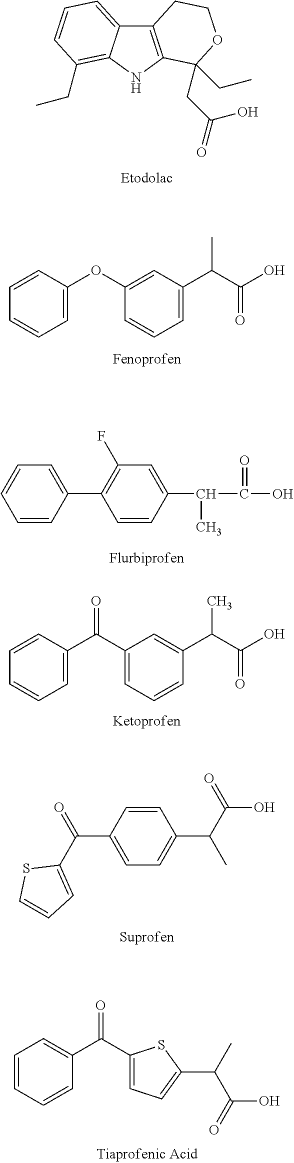 [SMILES: CCc1cccc2c3c([nH]c12)C(CC)(CC(=O)O)OCC3 ; CC(C(=O)O)c1cccc(Oc2ccccc2)c1 ; CC(C(=O)O)c1ccc(-c2ccccc2)c(F)c1 ; CC(C(=O)O)c1cccc(C(=O)c2ccccc2)c1 ; CC(C(=O)O)c1ccc(C(=O)c2cccs2)cc1 ; CC(C(=O)O)c1ccc(C(=O)c2ccccc2)s1]

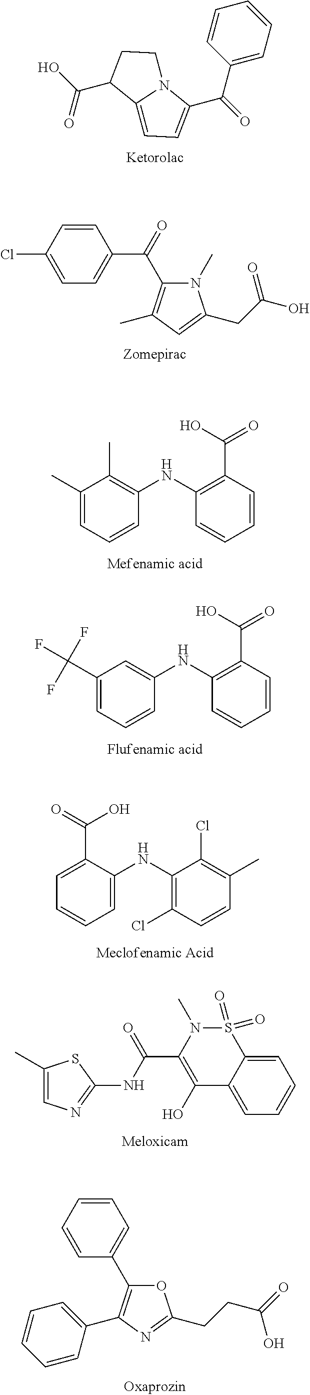

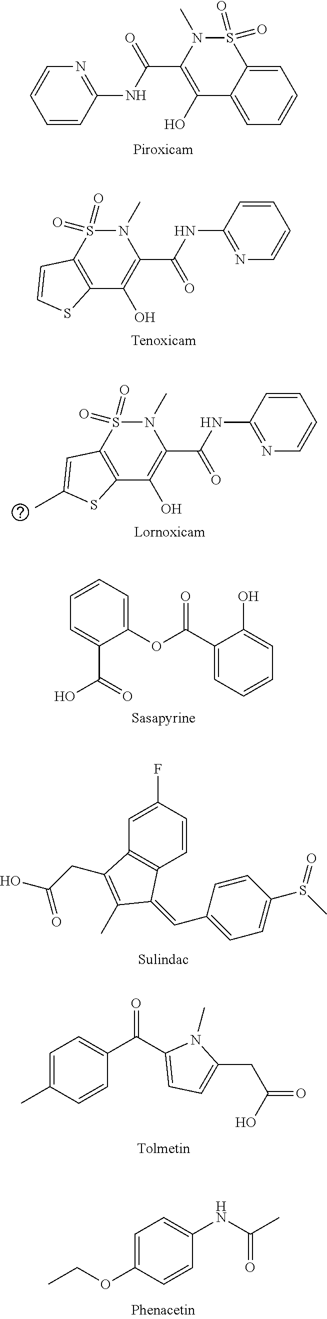

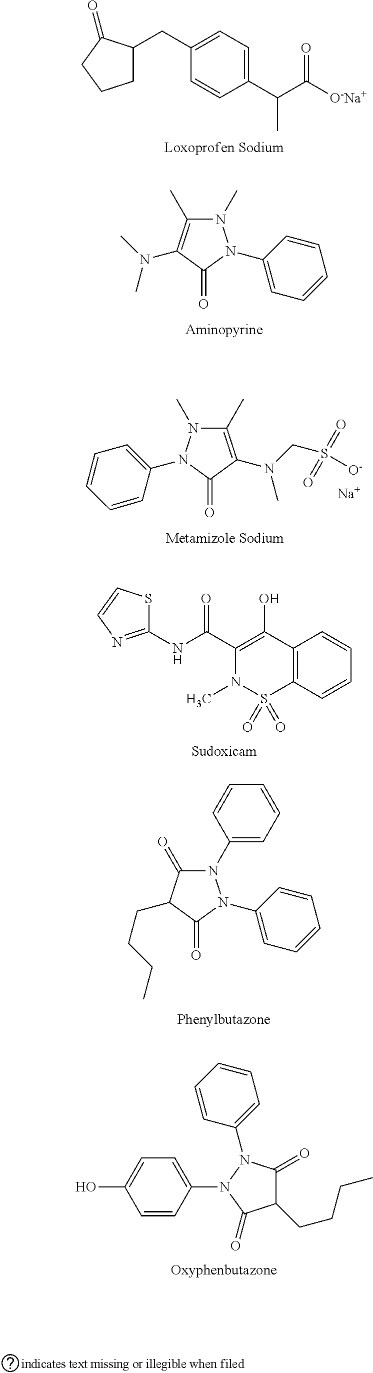

Non-steroidal analgesics mainly include:
1. Salicylic acids, the representative drug is Aspirin.
2. Acetanilides, the representative drug is Paracetamol.
3. Arylacetic acids, the representative drugs include Diclofenac, Indomethacin and Sulindac.
4. Arylpropionic acids, the representative drugs include Diclofenac, Naproxen and Flurbiprofen axetil.
5. Oxicams, the representative drugs include Flunoxicam, Piroxicam and Meloxicam.
6. Pyrazolones, the representative drugs include Phenylbutazone, Oxyphenbutazone and Metamizole.
7. Selective COX-2 inhibitors, the representative drugs include Celecoxib, Nimesulide, Etoricoxib and Parecoxib.

The nearly forty drugs listed above can all be classified into the above-mentioned seven categories.

The AKR1C3-activated anticancer prodrug compound is selected from the compounds having structural formulae (1)-(11):

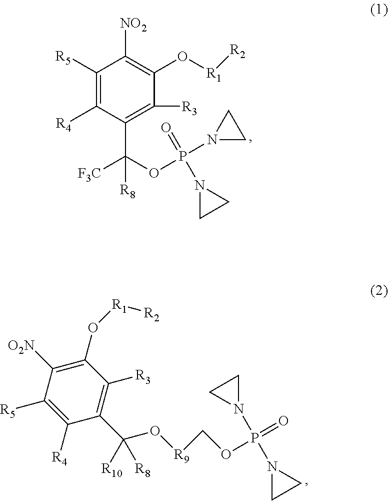

wherein, the definitions of R₁, R₂, R₃, R₄, R₅, R₈, R₉, and R₁₀ are described in the claims of Patent Application PCT/CN2020/089692 with Publication No. WO2020228685A1 (corresponding to Chinese Patent Application No. 2020800358890 with Publication No. CN113853379A), which describes the synthesis and preparation methods of specific compounds as well and is incorporated herein by reference in its entirety, with detailed definitions being:
wherein,
R₁ is C₆-C₁₀ aryl or Z-substituted aryl, 4-15-membered heterocycle or Z-substituted heterocycle, 5-15-membered heteroaryl or Z-substituted heteroaryl, or 7-15-membered fused ring or Z-substituted fused ring; R₂ is hydrogen, a halogen atom, cyano or isocyano, hydroxy, sulfhydryl, amino, OTs, OMS, C1-C₆ alkyl or Z-substituted alkyl, C₂-C₆ alkenyl or Z-substituted alkenyl, C₂-C₆ alkynyl or Z-substituted alkynyl, C₃-C₅ cycloalkyl or Z-substituted cycloalkyl, C₆-C₁₀ aryl or Z-substituted aryl, 4-15-membered heterocycle or Z-substituted heterocycle, 5-15-membered heteroaryl or Z-substituted heteroaryl, ether having from 1 to 6 carbon atoms or Z-substituted alkoxy having from 1 to 6 carbon atoms, —CONR⁶R⁷, —SO₂NR⁶R⁷, —SO₂R⁶, —OCOO—R⁶, —COOR⁶, —NR⁶COR⁷, —OCOR⁶, —NR⁶SO₂R⁷ or —NR⁶SO₂NR⁶R⁷, or R² together with the atom in the group R₁ to which it is bonded to form a 7-15-membered fused ring or Z-substituted fused ring;
R₃ is hydrogen, halogen, cyano or isocyano, hydroxy, sulfhydryl, amino, OTs, OLCMS, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, C₆-C₁₀ aryl or Z-substituted aryl, 4-15-membered heterocycle or Z-substituted heterocycle, 5-15-membered heteroaryl or Z-substituted heteroaryl, C₁-C₆ alkoxy or Z-substituted C₁-C₆ alkoxy, —CONR⁶R⁷, —SO₂NR⁶R⁷, —SO₂R⁶, —OCO—R⁶, —OCOO—R⁶, —COOR⁶, —NR⁶COR⁷, —OCOR⁶, or —NR⁶SO₂R⁷;
R₄ and R₅ are each independently hydrogen, a halogen atom, cyano or isocyano, hydroxy, sulfhydryl, amino, OTs, OLCMS, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, C₆-C₁₀ aryl or Z-substituted aryl, 4-15-membered heterocycle or Z-substituted heterocycle, 5-15-membered heteroaryl or Z-substituted heteroaryl, C₁-C₆ alkoxy or Z-substituted C₁-C₆ alkoxy, —CONR⁶R⁷, —SO₂NR⁶R⁷, —SO₂R⁶, —OCOO—R⁶, —COOR⁶, —NR⁶COR⁶, —OCOR⁶ or —NR⁶SO₂R⁷, or R₄ and R₅ together with the atom in the benzene ring to which they are bonded to form a 7-15-membered fused ring or Z-substituted fused ring;
R⁶ and R⁷ are each independently hydrogen, cyano or isocyano, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, C₆-C₁₀ aryl or Z-substituted aryl, 4-15-membered heterocycle or Z-substituted heterocycle, 5-15-membered heteroaryl or Z-substituted heteroaryl, or C₁-C₆ alkoxy or Z-substituted C₁-C₆ alkoxy, or R⁶ and R⁷ together with the atom to which they are bonded to form 5-7-membered heterocyclyl or Z-substituted 5-7-membered heterocyclyl;
R₈ and R₁₀ are each independently hydrogen, deuterium, aryl or Z-substituted aryl, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, and at least one of R₈ and R₁₀ must be hydrogen or deuterium; R₉ is substituted C₆-C₁₀ aryl which is substituted with at least one fluorine atom or nitro group, substituted 4-15-membered heterocycle which is substituted with at least one fluorine atom or nitro group, or substituted 5-15-membered heteroaryl which is substituted with at least one fluorine atom or nitro group; the substituent Z is a halogen atom, cyano or isocyano, hydroxy, sulfhydryl, amino, OTs, OMS, C₁-C₃ alkyl or substituted alkyl, C₁-C₃ alkoxy or substituted alkoxy, C₂-C₃ alkenyl or substituted alkenyl, C₂-C₃ alkynyl or substituted alkynyl, C₃-C₈ cycloalkyl or substituted cycloalkyl, an aromatic ring, heterocycle, a heteroaromatic ring and fused ring or a substituted aromatic ring, heterocycle, or a heteroaromatic ring and fused ring, the pattern of substitution being mono- or geminal di-substitution;
the substitution in the substituted C₆-C₁₀ aryl, substituted 4-15-membered heterocycle or substituted 5-15-membered heteroaryl in R₉ is a halogen atom, nitro, cyano or isocyano, hydroxy, amino, C₁-C₃ alkyl or alkoxy, alkenyl, alkynyl, cycloalkyl or benzene ring, substituted benzene ring, C₁-C₃ alkoxy or halogen atom-substituted alkoxy.

Regarding the typical exemplified compounds, please see the specific compounds listed in the Example section of Patent Application PCT/CN2020/089692 with Publication No. WO2020228685A1, which describes the preparation methods and spectral data of these compounds as well and is incorporated herein by reference in its entirety.

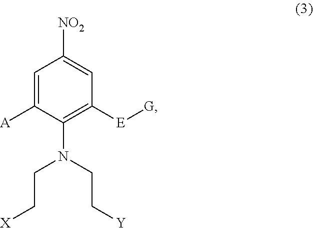

wherein, the definitions of A, E, G, X and Y are described in the claims of Patent Application PCT/NZ2019/050030 with Publication No. WO2019190331A1 (corresponding to Chinese Patent Application No. 2019800234236 with Publication No. CN111918864A), which describes the synthesis and preparation methods of specific compounds as well and is incorporated herein by reference in its entirety, with detailed definitions being:
wherein:
A is H, C₁-C₆ alkyl, C₁-C₆ alkenyl, C₁-C₆ alkynyl, CFH₂, CF₂H, CF₃, F, Cl, Br, I, OCF₃, COR or CON(R)₂;
E is SO or SO₂;
X is Cl, Br, I or OSO₂R;
Y is Cl, Br, I or OSO₂R;
each R is independently H or C₁-C₆ alkyl;
G is a radical group selected from the group consisting of Formulae (B)-(AA):

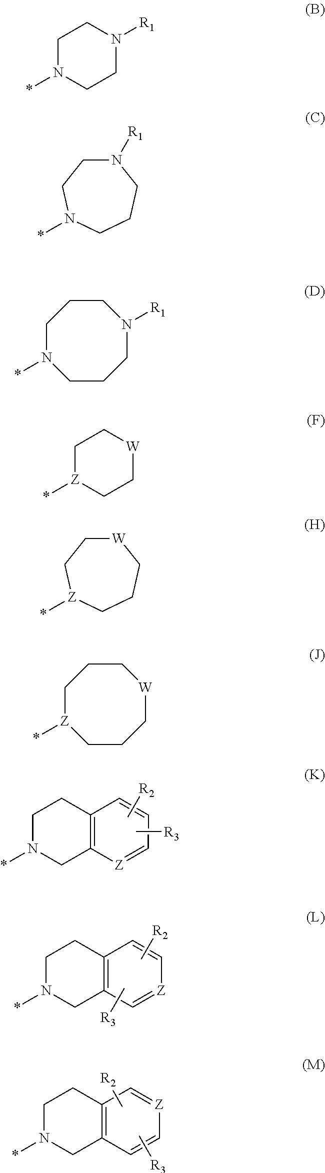

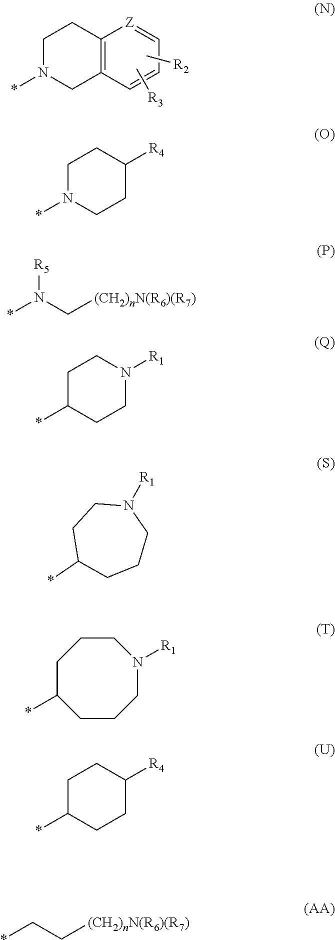

wherein:
R₁ is H, C₁-C₆ alkyl, CH₂(CH₂)ₙOH, CH₂CH(OH)CH₂OH, phenyl, pyridyl, benzyl, or pyridylmethyl, provided that when R₁ is phenyl, pyridyl, benzyl or pyridylmethyl, R₁ is optionally substituted at any available position with C₁-C₆ alkyl, C₁-C₆ alkenyl, C₁-C₆ alkynyl, OR₆, N(R₆)(R₇), CFH₂, CF₂H, CF₃, F, Cl, Br, I, OCF₃, COR₆, CON(R₆)(R₇), SOR₆, SON(R₆)(R₇), SO₂R₆, SO₂N(R₆)(R₇), CN, or NO₂;
R₂ and R₃ are each independently H, C₁-C₆ alkyl, C₁-C₆ alkenyl, C₁-C₆ alkynyl, OR₆, N(R₆)(R₇), CFH₂, CF₂H, CF₃, F, Cl, Br, I, OCF₃, COR₆, CON(R₆)(R₇), SOR₆, SON(R₆)(R₇), SO₂R₆, SO₂N(R₆)(R₇), CN or NO₂;
R₄ is N(R₆)(R₇), OH, OCH₂(CH₂)ₙN(R₆)(R₇) or CH₂(CH₂)ₙN(R₆)(R₇);
R₅ is H or a C₁-C₆ alkyl group;
R₆ and R₇ are each independently H or C₁₋₆ alkyl, or R₆ and R₇ together form a substituted or unsubstituted 5-membered or 6-membered heterocycle;
Z is CH or N;
W is CH₂, O, S, SO or SO₂;
n is 0 to 6;
* represents a point of attachment to Formula (I).

Regarding the typical exemplified compounds, please see the specific compounds listed in the Example section of Patent Application PCT/NZ2019/050030 with Publication No. WO2019190331A1 (corresponding to Chinese Patent Application No. 2019800234236 with Publication No. CN111918864A), which describes the preparation methods and spectral data of these compounds as well and is incorporated herein by reference in its entirety.

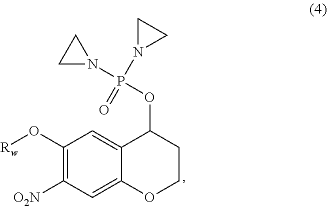

wherein, the definition of Rw is described in the claims of Patent Application PCT/CN2020/120281 with Publication No. WO2021068952A1 (corresponding to Chinese Patent Application No. 202080071652.8 with Publication No. CN114555574A), which describes the synthesis and preparation methods of specific compounds as well and is incorporated herein by reference in its entirety, with detailed definitions being:
Rw is;

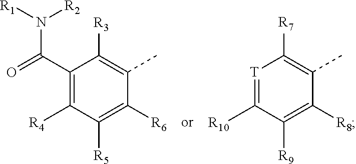

R₁ is H, C₁₋₆ alkyl, C₃₋₆ cycloalkyl, 4-6-membered heterocycloalkyl, 5-6-membered heteroaryl or phenyl, wherein the C₁₋₆ alkyl, C₃₋₆ cycloalkyl, 4-6-membered heterocycloalkyl, 5-6-membered heteroaryl and phenyl are optionally substituted with 1, 2 or 3 R^{a} groups;
each R^{a} is independently H, F, Cl, Br, I, —CN, —OH, C₁₋₃ alkoxy or C₁₋₃ alkyl;
R₂ is H or C₁₋₆ alkyl;
or R₁ and R₂, together with the N atom to which they are attached, to form a 4-6-membered heterocycloalkyl, wherein the 4-6-membered heterocycloalkyl is optionally substituted with 1, 2 or 3 R^{b} groups;
each R^{b} is independently H, F, Cl, Br, I, —CN, —OH, —NH₂, —OCH₃, —OCH₂CH₃, —CH₃ or —CH₂CH₃;
R₃ is H, F, Cl, Br, I, —OH, —NH₂, C₁₋₃ alkoxy or C₁₋₃ alkyl;
or R₂ and R₃ are attached together to make the structural unit

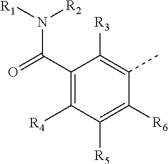

to be

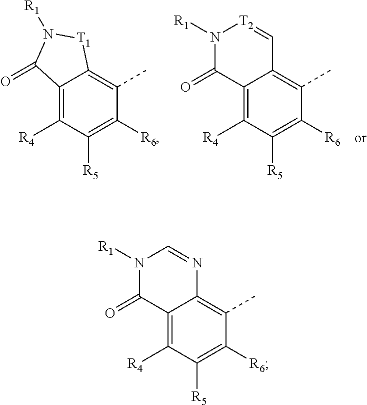

T₁ is −(CR^{c}R^{d})ₘ— or —(CR^{c}R^{d})ₙ—O—;
m is 1, 2 or 3;
n is 1 or 2;
T₂ is N or CH;
R^{c} and R^{d} are each independently H, F, C₁₋₃ alkyl or C₁₋₃ alkoxy;
R₄, R₅ and R₆ are each independently H, F, Cl, Br, I, C₁₋₃ alkyl or C₁₋₃ alkoxy;
T is N or CH;
R₇ and R₈ are each independently H, F, Cl, Br or I;
R₉ and R₁₀ are each independently H, F, Cl, Br, I, —CN; or
the 4-6-membered heterocycloalkyl and 5-6-membered heteroaryl each contain 1, 2, 3 or 4 heteroatoms independently selected from the group consisting of N, —O— and —S—.

Regarding the typical exemplified compounds, please see the specific compounds listed in the Example section of Patent Application PCT/CN2020/120281 with Publication No. WO2021068952A1 (corresponding to Chinese Patent Application No. 202080071652.8 with Publication No. CN114555574A), which describes the preparation methods and spectral data of these compounds as well and is incorporated herein by reference in its entirety.

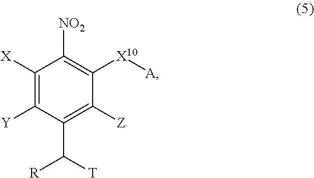

wherein, the definitions of X, Y, Z, R, T, A and X¹⁰ are described in the claims of Patent Application PCT/US2016/021581 with Publication No. WO2016145092A1 (corresponding to Chinese Patent Application No. 2016800150788 with Publication No. CN107530556A), which describes the synthesis and preparation methods of specific compounds as well and is incorporated herein by reference in its entirety, with detailed definitions being:
X¹⁰ is O, S, SO, or SO₂;
A is C₆-C₁₀ aryl, 5-15-membered heteraryl or —N═CR¹R²;
R¹ and R² are each independently hydrogen, C₁-C₆ alkyl, C₃-C₈ cycloalkyl, C₆-C₁₀ aryl, 4-15-membered heterocycle, ether, —CONR¹³R¹⁴ or —NR¹³COR¹⁴;
X, Y and Z are independently hydrogen, CN, a halogen group, C₁-C₆ alkyl, C₂-C₆ alkenyl, C₂-C₆ alkynyl,
C₃-C₈ cycloalkyl, C₆-C₁₀ aryl, 4-15-membered heterocycle, ether, —CONR¹³R¹⁴ or —NR¹³COR¹⁴;
R is hydrogen, C₁-C₆ alkyl, C₂-C₆ alkenyl, C₂-C₆ alkynyl, C₃-C₈ cycloalkyl, C₆-C₁₀ aryl, 4-15-membered heterocycle, ether, —CONR¹³R¹⁴ or —NR¹³COR¹⁴;
R¹³ and R¹⁴ are each independently hydrogen, C₁-C₆ alkyl, C₃-C₈ cycloalkyl, C₆-C₁₀ aryl, 4-15-membered heterocycle or ether;
T comprises a phosphoramidate alkylating agent comprising one or more Z⁵—X⁵—Y⁵ moieties bonded to an —O—P(Z¹) moiety, wherein Z⁵ is a heteroatom such as nitrogen, sulfur or oxygen, X⁵ is substituted or unsubstituted ethylene, Y⁵ is a halogen group or another leaving group, or Z⁵—X⁵—Y⁵ together form an aziridinyl (NCH₂CH₂) moiety, and Z¹ is O or S; and
wherein the alkyl, alkenyl, alkynyl, cycloalkyl, aryl, heterocyclyl, heteroaryl, ether groups are substituted or unsubstituted.

Regarding the typical exemplified compounds, please see the specific compounds listed in the Example section of Patent Application PCT/US2016/021581 with Publication No. WO2016145092A1 (corresponding to Chinese Patent Application No. 2016800150788 with Publication No. CN107530556A), which describes the preparation methods and spectral data of these compounds as well and is incorporated herein by reference in its entirety.

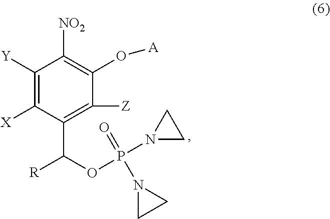

wherein:
A is a substituted or unsubstituted C₆-C₁₀ aryl, a biaryl or a substituted biaryl, a 5-15-membered heteroaryl, or —N═CR¹R²; wherein the substituent is selected from the group consisting of a halogen group, —CN, —NO₂, —O—(CH₂)—O—, —CO₂H and salt thereof, —OR¹⁰⁰, —CO₂R¹⁰⁰, —CONR¹⁰¹R¹⁰², —NR¹⁰¹R¹⁰², —NR¹⁰¹O₂R¹⁰⁰, —SO₂R¹⁰⁰, —SO₂NR¹⁰¹R¹⁰², C₁-C₆ alkyl, and C₃-C₁₀ heterocyclyl;
wherein R¹⁰⁰, R¹⁰¹ and R¹⁰² are each independently hydrogen, C₁-C₈ alkyl, or C₆-C₁₂ aryl; or R¹⁰¹ and R¹⁰² together with the nitrogen atom to which they are attached form a 5-7-membered heterocycle; wherein the alkyl group and the aryl group are each substituted by 1-3 halogen groups or 1-3 C₁-C₆ alkyl groups;
R¹ and R² are each independently phenyl or methyl;
X, Y and Z are each independently hydrogen or a halogen group; and
R is hydrogen or C₁-C₆ alkyl or halogen-substituted alkyl.

Regarding the typical exemplified compounds, please see the specific compounds listed in the Example section of Patent Application PCT/US2016/021581 with Publication No. WO2016145092A1 (corresponding to Chinese Patent Application No. 2016800150788 with Publication No. CN107530556A), which describes the preparation methods and spectral data of these compounds as well and is incorporated herein by reference in its entirety.

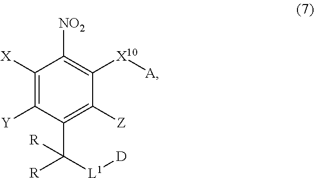

wherein, the definitions of X, Y, Z, R, D, L¹, A and X¹⁰ are described in the claims of Patent Application PCT/US2016/025665 with Publication No. WO2016161342A3 (corresponding to Chinese Patent Application No. 2016800200132 with Publication No. CN108136214A), which describes the synthesis and preparation methods of specific compounds as well and is incorporated herein by reference in its entirety, with detailed definitions being:
X¹⁰ is O, S, SO, or SO₂;
A is C₆-C₁₀ aryl, 5-15-membered heteroaryl, or —N═CR¹R²;
R¹ and R² are each independently hydrogen, C₁-C₆ alkyl, C₃-C₈ cycloalkyl, C₆-C₁₀ aryl, 4-15-membered heterocycle, 5-15-membered heteroaryl, ether, —CONR¹³R¹⁴, or —NR¹³COR¹⁴;
X, Y, and Z are each independently hydrogen, CN, a halogen group, C₁-C₆ alkyl, C₂-C₆ alkenyl, C₂-C₆ alkynyl, C₃-C₈ cycloalkyl, C₆-C₁₀ aryl, 4-15-membered heterocycle, 5-15-membered heteroaryl, ether, —CONR¹³R¹⁴, or —NR¹³COR¹⁴;
each R is independently hydrogen, C₁-C₆ alkyl, C₂-C₆ alkenyl, C₂-C₆ alkynyl, C₃-C₈ cycloalkyl, C₆-C₁₀ aryl, 4-15-membered heterocycle, 5-15-membered heteroaryl, ether, —CONR¹³R¹⁴, or —NR¹³COR¹⁴;
R¹³ and R¹⁴ are each independently hydrogen, C₁-C₆ alkyl, C₃-C₈ cycloalkyl, C₆-C₁₀ aryl, 4-15-membered heterocycle, 5-15-membered heteroaryl or ether;
wherein the definition of L¹ and D are as follows:
L¹ is selected from the group consisting of

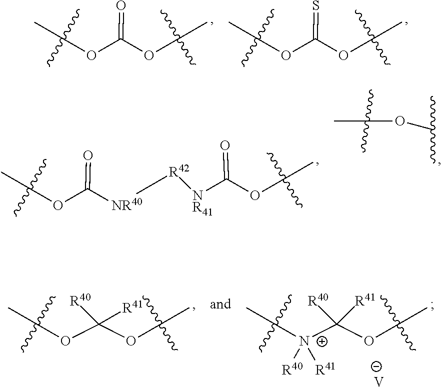

R⁴⁰ and R⁴¹ are independently hydrogen, C₁-C₆ alkyl, C₂-C₆ alkenyl, C₂-C₆ alkynyl, C₃-C₈ cycloalkyl, C₆-C₁₀ aryl, 4-15-membered heterocycle, or 5-15-membered heteroaryl;
R⁴² is C₂-C₃ alkylene or heteroalkylene optionally substituted with 1-3 C₁-C₆ alkyl groups;
V (−) is any anion, preferably a pharmaceutically acceptable anion;
D is a moiety that makes D-OH an anti-cancer drug, wherein OH is an aliphatic or a phenolic hydroxy group or is an OH moiety attached to a phosphorous atom as provided herein; or
L₁ is:

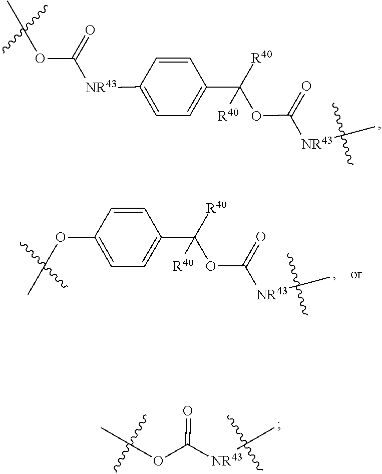

R⁴⁰ is as defined above, R⁴³ is hydrogen or together with D forms a heterocycle, and the phenylene moiety is optionally substituted; and
D is a moiety that makes D-NR⁴³H an anti-cancer drug; or
L¹ is a bond, —O—C(R⁴⁰R⁴¹)—, —O—C(R⁴⁰R⁴¹)—NR⁴⁰R⁴¹(+)—C(R⁴⁰R⁴¹)— or

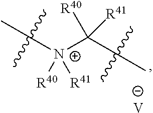

wherein R⁴⁰, R⁴¹, and V are as defined above; and
D is an anti-cancer drug containing a tertiary or a secondary nitrogen atom, wherein the tertiary or the secondary nitrogen atom is bonded to L¹; and
wherein the alkyl, alkenyl, alkynyl, cycloalkyl, aryl, heterocycle, heteroaiyl, ether are optionally substituted.

Regarding the typical exemplified compounds, please see the specific compounds listed in the Example section of Patent Application PCT/US2016/025665 with Publication No. WO2016161342A3 (corresponding to Chinese Patent Application No. 2016800200132 with Publication No. CN108136214A), which describes the preparation methods and spectral data of these compounds as well and is incorporated herein by reference in its entirety.

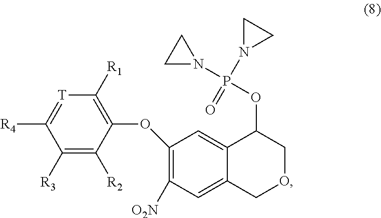

wherein, the definitions of R₁, R₂, R₃, R₄, and T are described in the claims of Patent Application PCT/CN2021/118597 with Publication No. WO2022057838A1, which describes the synthesis and preparation methods of specific compounds as well and is incorporated herein by reference in its entirety, with detailed definitions being:
T is N or CH;
R₁ and R₂ are each independently H, F, Cl, Br, I, or C₁₋₃ alkyl, wherein the C₁₋₃ alkyl is optionally substituted with 1, 2, or 3 Rₐ groups;
each Rₐ is independently F, Cl, Br, I, —CN, —OH, or —NH₂;
R₃ and R₄ are each independently H, F, Cl, Br, I, CN, C₁₋₃ alkyl, C₁₋₃ alkoxy,

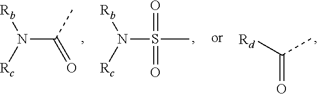

wherein the C₁₋₃ alkyl is optionally substituted with 1, 2, or 3 Rₑ groups;
R_{b} and R_{c} are each independently H, —CH₃, —CH₂CH₃, —(CH₂)₂CH₃, or —CH(CH₃)₂;
R_{d} is —CH₃, —CH₂CH₃, —(CH₂)₂CH₃, or —CH(CH₃)₂;
each Rₑ is independently F, Cl, Br, I, —CN, —OH, or —NH₂.

Regarding the typical exemplified compounds, please see the specific compounds listed in the Example section of Patent Application PCT/CN2021/118597 with Publication No. WO2022057838A1, which describes the preparation methods and spectral data of these compounds as well and is incorporated herein by reference in its entirety.

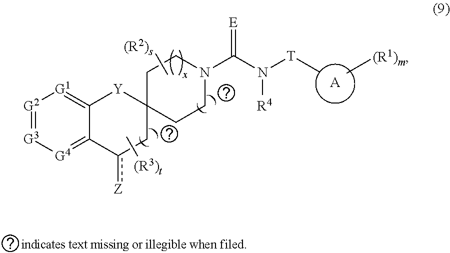

wherein, the definitions of R¹, R², R³, R⁴, G¹, G², G³, G⁴, E, T, Y, Z, m, n, s, t, v, w, and Ring A are described in the claims of Patent Application CN202210585771.6 with Publication No. CN115403579A, which describes the synthesis and preparation methods of specific compounds as well and is incorporated herein by reference in its entirety, with detailed definitions being:
G¹, G², G³, and G⁴ are the same or different, and are each independently CR⁵ or an N atom;
each R⁵ is the same or different, and each is independently selected from the group consisting of hydrogen, halogen, alkyl, alkenyl, alkynyl, alkoxy, hydroxyl, cyano, amino, nitro, —NR^{a}R^{b}, —C(O)NR^{a}R^{b}, cycloalkyl, heterocyclyl, aryl, and heteroaryl, wherein the alkyl, alkenyl, alkynyl, alkoxy, cycloalkyl, heterocyclyl, aryl, and heteroaryl are each independently optionally substituted with one or more substituents selected from the group consisting of halogen, alkyl, haloalkyl, alkoxy, haloalkoxy, hydroxyalkyl, hydroxyl, oxo, cyano, amino, nitro, cycloalkyl, heterocyclyl, aryl, and heteroaryl;
Y is selected from the group consisting of —(C(R^{y2}R^{y3}))_{f}—NR^{y1}—, —(C(R^{y2}R^{y3}))_{g}—O—, —(C(R^{y2}R^{y3}))ₕ—S—, —(C((R^{y2}R^{y3}))ₕ—S(O)—, —(C(R^{y2}R^{y3}))ₕ—S(O)₂—, —C(R^{y2}R^{y3})—, —NR^{y1}—(C(R^{y2}R^{y3}))_{f}—, —O—(C(R^{y2}R^{y3}))_{g}—, —S—(C(R^{y2}R^{y3}))ₕ—, —S(O)—(C(R^{y2}R^{y3}))ₕ—, and —S(O)₂—(C(R^{y2}R^{y3}))ₕ—;
R^{y1} is selected from the group consisting of hydrogen, alkyl, haloalkyl, hydroxyalkyl, cycloalkyl, and heterocyclyl;
R^{y2} and R^{y3} are the same or different, and are each independently selected from the group consisting of hydrogen, halogen, alkyl, haloalkyl, hydroxyalkyl, cycloalkyl, and heterocyclyl;
or R^{y2} and R^{y3} together form ═O;
Z is O or OH:
[symbol] is a single bond or double bond, and Z is OH when [symbol] is a single bond, and Z is O when [symbol] is a double bond;
E is selected from the group consisting of NH, O, and S;
T is selected from the group consisting of —C(R^{T1}R^{T2})—, —NR^{T3}—, and —O—;
R^{T1} and R^{T2} are the same or different, and are each independently selected from the group consisting of hydrogen, deuterium, halogen, alkyl, haloalkyl, hydroxyalkyl, cycloalkyl, and heterocyclyl;
or R^{T1} and R^{T2} together with the carbon atom to which they are attached form cycloalkyl or heterocyclyl, and the cycloalkyl or heterocyclyl is each independently optionally substituted with one or more substituents selected from the group consisting of halogen, alkyl, and hydroxyl;
R^{T3} is selected from the group consisting of hydrogen, alkyl, haloalkyl, hydroxyalkyl, cycloalkyl, and heterocyclyl;
Ring A is 6-10-membered aryl or 5-10-membered heteroaryl;
each R¹ is the same or different, and each is independently selected from the group consisting of hydrogen, deuterium, halogen, alkyl, alkenyl, alkynyl, alkoxy, hydroxyl, cyano, —NR^{a}R^{b}, —C(O)NR^{a}R^{b}, —S(O)NR^{a}R^{b}, —S(O)₂NR^{a}R^{b}, —S(O)R^{c}, —S(O)₂R^{c}, —B(OR^{d})₂, nitro, cycloalkyl, heterocyclyl, aryl, and heteroaryl, wherein the alkyl, alkenyl, alkynyl, alkoxy, cycloalkyl, heterocyclyl, aryl, and heteroaryl are each independently optionally substituted with one or more substituents selected from the group consisting of halogen, alkyl, haloalkyl, alkoxy, haloalkoxy, hydroxyalkyl, hydroxyl, oxo, cyano, —NR^{a}R^{b}, —C(O)NR^{a}R^{b}, —S(O)NR^{a}R^{b}, —S(O)₂NR^{a}R^{b}, —S(O)R^{c}, —S(O)₂R^{c}, —B(OR^{d})₂, nitro, cycloalkyl, heterocyclyl, aryl, and heteroaryl;
R^{a} and R^{b} are the same or different, and are each independently selected from the group consisting of hydrogen, alkyl, haloalkyl, hydroxyl, hydroxyalkyl, —C(O)R′, cycloalkyl, and heterocyclyl; or R^{a} and R^{b} together with the nitrogen atom to which they are attached form a cycloalkyl or heterocyclyl, wherein the cycloalkyl or heterocyclyl is optionally substituted with one or more substituents selected from the consisting of halogen, alkyl, haloalkyl, alkoxy, haloalkoxy, hydroxyalkyl, hydroxyl, cyano, amino, nitro, cycloalkyl, heterocyclyl, aryl, and heteroaryl;
R^{c} is selected from the group consisting of alkyl, cycloalkyl, heterocyclyl, aryl, and heteroaryl, which are optionally substituted with one or more substituents selected from the group consisting of halogen, alkyl, haloalkyl, alkoxy, haloalkoxy, hydroxyalkyl, hydroxyl, cyano, amino, nitro, cycloalkyl, heterocyclyl, aryl, and heteroaryl;
R^{d} is hydrogen or C₁₋₆ alkyl;
R^{e} is selected from the group consisting of alkyl, alkoxy, cycloalkyl, heterocyclyl, aryl, and heteroaryl, which are optionally substituted with one or more substituents selected from the group consisting of halogen, alkyl, haloalkyl, alkoxy, haloalkoxy, hydroxyalkyl, hydroxyl, cyano, amino, nitro, cycloalkyl, heterocyclyl, aryl, and heteroaryl;
each R² is the same or different, and each is independently selected from the group consisting of hydrogen, deuterium, halogen, alkyl, haloalkyl, alkoxy, haloalkoxy, hydroxyalkyl, oxo, hydroxyl, cyano, amino, nitro, cycloalkyl, heterocyclyl, aryl, and heteroaryl;
each R³ is the same or different, and each is independently selected from the group consisting of hydrogen, deuterium, halogen, alkyl, haloalkyl, alkoxy, haloalkoxy, hydroxyalkyl, oxo, hydroxyl, cyano, amino, nitro, cycloalkyl, heterocyclyl, aryl, and heteroaryl;
R⁴ is selected from the group consisting of hydrogen, alkyl, haloalkyl, hydroxyl, and hydroxyalkyl;
n is 0, 1, 2, or 3;
v is 0, 1, or 2;
w is 0, 1, or 2;
f is 0, 1, or 2;
g is 0, 1, or 2;
h is 0, 1, or 2;
m is 0, 1, 2, 3, 4, or 5;
s is 0, 1, 2, 3, 4, 5, 6, 7, or 8;
t is 0, 1, 2, 3, 4, 5, or 6;
provided that
when Y is —O— and E is O, Ring A is phenyl or 5-6-membered heteroaryl, and G³ is CR⁵ or N, and R⁵ is not hydrogen;
when Y is —O— and E is S, Ring A is phenyl or 5-6-membered heteroaryl;
when G¹, G², G³, and G⁴ are all CR⁵, Y is NR^{y1}, n, v, and w are all 1, and E is O, 1) T is not CH₂ or CD₂; 2) at least one of R² or R³ is deuterium; 3) R⁴ is selected from the group consisting of alkyl, haloalkyl, hydroxyl, and hydroxyalkyl; 4) one of R¹ is 3-8-membered cycloalkyl or 5-8-membered heterocyclyl, wherein 3-8-membered cycloalkyl or 5-8-membered heterocyclyl is optionally substituted with one or more substituents selected from the group consisting of halogen, alkyl, haloalkyl, alkoxy, haloalkoxy, hydroxyalkyl, hydroxyl, oxo, cyano, amino, nitro, cycloalkyl, heterocycle, aryl, and heteroaryl; 5) Ring A is R^{d}, which is hydrogen or C₁₋₆ alkyl.

Regarding the typical exemplified compounds, please see the specific compounds listed in the Example section of Chinese Patent Application CN202210585771.6 with Publication No. CN115403579A, which describes the preparation methods and spectral data of these compounds as well and is incorporated herein by reference in its entirety.

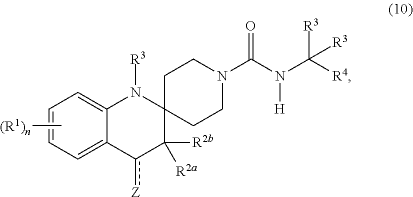

or a pharmaceutically acceptable salt thereof, wherein, the definitions of R¹, R^{2a}, R^{2b}, R³, R⁴, R⁵, n, and Z are described in the claims of Patent Application PCT/IB2020/057285 with Publication No. WO2021005586A1 (corresponding to Chinese Patent Application No. CN202080053804.1 with Publication No. CN114206870A), which describes the synthesis and preparation methods of specific compounds as well and is incorporated herein by reference in its entirety, with detailed definitions being:
[symbol] is a single bond or double bond;
Z is OH when [symbol] is a single bond; or is O when [symbol] is a double bond;
each R¹ is independently selected from the group consisting of (C₁-C₆)-alkyl, (C₁-C₆)-alkoxy, (C₀-C₄)-alkyl-N(R⁸)₂, and a halogen group;
R^{2a} and R^{2b} are each independently selected from the group consisting of H, (C₁-C₆)-alkyl, and a halogen group;
each R³ is independently selected from the group consisting of H and a halogen group;
R⁴ is selected from the group consisting of aryl; 5-6-membered heteroaryl containing 1, 2, 3, or 4 heteroatoms independently selected from the group consisting of N, O, and S; and 9-10-membered fused bicyclic heteroaryl containing 1, 2, 3, or 4 heteroatoms independently selected from the group consisting of N, O, and S; wherein any one of the above is optionally substituted with one or more R⁶ groups;
R⁵ is selected from the group consisting of H, (C₁-C₆)-alkyl, (C₂-C₆)-alkenyl, (C₀-C₄)-alkyl-OR⁸, (C₁-C₄)-alkyl-(C₃-C₁₀)-cycloalkyl, halo-(C₁-C₆)-alkyl, (C₂-C₃)-alkynyl, and (C₁-C₄)-alkyl-N(R¹⁰)₂;
each R⁶ is independently selected from the group consisting of a halogen group, (C₁-C₆)-alkyl, (C₁-C₆)-alkoxy, halo-(C₁-C₆)-alkyl, OH, aryl, 3-6-membered heterocycle, 5-6-membered heteroaryl, (C₀-C₄)-alkyl-S(O)ₘ—(C₁-C₆)-alkyl, halo-(C₁-C₆)-alkoxy, (C₀-C₄)-alkyl-S(O)ₘN(R⁸)₂, (C₀-C₄)-alkyl-N(R⁸)₂, (C₀-C₄)-alkyl-(CO)OR⁷, N(R⁸)S(O)ₘ—(C₁-C₆)-alkyl, N(R⁸)S(O)ₘ—(C₃-C₆)-cycloalkyl, OP(O)(OH)₂, (C₀-C₃)-alkyl-(CO)NHR¹¹, (C₀-C₃)-alkyl-OR⁷, and (C₃-C₁₀)-cycloalkyl; wherein each R⁶ is optionally substituted with one to three R⁹ groups when not being a halogen group, OH, or OP(O)(OH)₂; or two adjacent R⁶ groups together with the atoms to which they are attached form a 5-7-membered heterocycle or (C₅-C₈)-cycloalkyl;
each R⁷ and R⁵ are independently selected from the group consisting of H and (C₁-C₆)-alkyl optionally substituted with one to three R⁹ groups;
each R⁹ is independently selected from the group consisting of a halogen group; —OH; amino, (C₁-C₄)-alkylamino, di-(C₁-C₄)-alkylamino, OP(O)(OH)₂; (C₁-C₆)-alkyl; (C₁-C₃)-alkynyl; (C₁-C₆)-alkoxy; halo-(C₁-C₆)-alkyl; (C₀-C₄)-alkyl-S(O)ₘ—(C₁-C₆)-alkyl; halo-(C₁-C₆)-alkoxy; 3-6-membered heterocycle optionally substituted with oxo (═O); (C₀-C₄)-alkyl-S(O)ₘN(R¹⁰)₂; (C₀-C₄)-alkyl-(CO)R¹⁰; (C₀-C₄)-alkyl-(CO)OR¹⁰; (C₀-C₄)-alkyl-NR¹⁰S(O)ₘ—(C₁-C₆)-alkyl; (C₀-C₄)-alkyl-OR¹⁰; (C₀-C₄)-alkyl-N(R¹⁰)₂; (C₀-C₄)-alkyl-CN; (C₀-C₄)-alkyl-N(R¹⁰)₂; and (C₀-C₄)-alkyl-(CO)N(R¹⁰)₂;
each R¹⁰ is independently selected from the group consisting of H; (C₁-C₆)-alkyl; and 3-6-membered heterocycle, wherein the 3-6-membered heterocycle is optionally substituted with one or more of (C₁-C₆)-alkyl and oxo (═O);
each R¹¹ is selected from the group consisting of H, 4-6-membered heterocycle optionally substituted with one to four R¹² groups, (C₃-C₆)-cycloalkyl optionally substituted with one to four R¹² groups, (C₀-C₃)-alkyl-(C₃-C₆)-cycloalkyl-(C₁-C₃)-alkyl optionally substituted with halogen, CH₂-aryl optionally substituted with one to three R¹² groups, (C₁-C₆)-alkyl, (C₂-C₆)-alkenyl, and (C₂-C₆)-alkynyl, wherein each one of the (C₁-C₆)-alkyl, (C₂-C₆)-alkenyl, and (C₂-C₆)-alkynyl is optionally substituted with one or more R¹³ groups;
each R¹² is independently selected from the group consisting of OH, (C₁-C₃)-alkoxy, NH₂, and (C₁-C₃)-alkyl optionally substituted with one or more OH groups;
each R¹³ is independently selected from the group consisting of a halogen group, OH, amino, (C₁-C₄)-alkylamino, di-(C₁-C₄)-alkylamino, (C₁-C₃)-alkoxy, and C(O)—(C₃-C₈)-cycloalkyl;
m is 0, 1, or 2; and
n is 0, 1, or 2.

Regarding the typical exemplified compounds, please see the specific compounds listed in the Example section of Patent Application PCT/IB2020/057285 with Publication No. WO2021005586A1 (corresponding to Chinese Patent Application No. CN202080053804.1 with Publication No. CN114206870A), which describes the preparation methods and spectral data of these compounds as well and is incorporated herein by reference in its entirety.

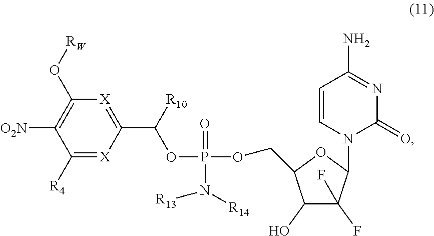

or a pharmaceutically acceptable salt thereof,
wherein, the definitions of R_{w}, X, R₄, R₁₀, R₁₃, and R₁₄ are described in the claims of Patent Application PCT/CN2022/098082 with Publication No. WO2022258043A1, which describes the synthesis and preparation methods of specific compounds as well and is incorporated herein by reference in its entirety, with detailed definitions being:
two X groups are each independently CR¹⁵ or N;
R₁₃ and R₁₄ are each independently hydrogen; C₁-C₆ alkyl; cycloalkyl; alkenyl; alkynyl; C₆-C₂₀ aryl; 5-20-membered heterocyclyl; halogenated C₁-C₆ alkyl, cycloalkyl, alkenyl, or alkynyl; halogenated C₆-C₂₀ aryl; or halogenated 5-20-membered heterocyclyl; and R₁₃ and R₁₄ are not hydrogen at the same time; R₁₀ is hydrogen; C₁-C₆ alkyl; cycloalkyl; alkenyl; alkynyl; C₆-C₂₀ aryl; 5-20-membered heterocyclyl; halogenated C₁-C₆ alkyl, cycloalkyl, alkenyl, or alkynyl; halogenated C₆-C₂₀ aryl; or halogenated 5-20-membered heterocyclyl;
or R₁₀ and R₁₃ or R₁₄ may be connected to form a 5-9-membered ring under the condition that they meet the above definitions for R₁₀, R₁₃ and R₁₄;
R₄ and R¹⁵ are each independently hydrogen; halogen; C₁-C₆ alkyl; cycloalkyl; alkenyl; alkynyl; alkoxy; cyano; 5-20-membered heterocyclyl; C₆-C₂₀ aryl; or halogenated C₁-C₆ alkyl, cycloalkyl, alkenyl, alkynyl, alkoxy, 5-20-membered heterocyclyl, or C₆-C₂₀ aryl;
or R₁₀ and R¹⁵ may form a 4-12-membered cyclic hydrocarbon or heterocycle under the condition that they meet the above definitions for R₁₀ and R¹⁵;
R_{W} is

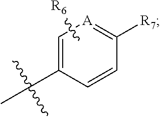

A is CR¹⁶ or N, wherein the position of A is variable on the ring;
R¹⁶ is hydrogen; C₁-C₆ alkyl; cycloalkyl; alkenyl; alkynyl; C₆-C₂₀ aryl; 5-20-membered heterocyclyl; halogenated C₁-C₆ alkyl, cycloalkyl, alkenyl, or alkynyl; halogenated C₆-C₂₀ aryl; or halogenated 5-20-membered heterocyclyl;
R₆ and R₇ meet the following conditions:
R₆ and R₇ are each independently hydrogen; halogen; cyano; hydroxyl; C₁-C₆ alkyl, cycloalkyl, alkenyl, alkynyl, alkoxy, 5-20-membered heterocyclyl, or C₆-C₂₀ aryl; or halogenated C₁-C₆ alkyl, cycloalkyl, alkenyl, alkynyl, alkoxy, 5-20-membered heterocyclyl, or C₆-C₂₀ aryl; or cyano-substituted C₁-C₆ alkyl, cycloalkyl, alkenyl, alkynyl, alkoxy, 5-20-membered heterocyclyl, or C₆-C₂₀ aryl; or hydroxyl-substituted C₁-C₆ alkyl, cycloalkyl, alkoxy, 5-20-membered heterocyclyl, or C₆-C₂₀ aryl; or —CONR¹¹R¹²; or —CH₂NR¹¹R¹²;
or R₆ and R₇ are connected to form
a 5-8-membered single heterocycle or fused heterocycle containing at least one of N or S or O or containing two or three of N, S, and O simultaneously;
or a 5-8-membered single heterocycle or fused heterocycle containing at least one of N or S or O or containing two or three of N, S, and O simultaneously, which is substituted with C₁-C₆ alkyl;
R₆ and CR¹⁶ may be connected to form a 5-9-membered ring, heterocycle, or aromatic heterocycle;
R¹¹ and R¹² meet the following conditions:
R¹¹ and R¹² are each independently C₁-C₆ alkyl or halogenated C₁-C₆ alkyl, or R¹¹ and R¹² form a 5-7-membered ring with N from —CONR¹¹R¹², or form a 5-7-membered ring with N from —CH₂NR¹¹R¹² under the condition that they meet the above definitions for R¹¹ and R¹².

Regarding the typical exemplified compounds, please see the specific compounds listed in the Example section of Patent Application PCT/CN2022/098082 with Publication No. WO2022258043A1, which describes the preparation methods and spectral data of these compounds as well and is incorporated herein by reference in its entirety.

Further, the AKR1C3 activated anticancer prodrug compound is selected from the compounds with the following structural formulae:

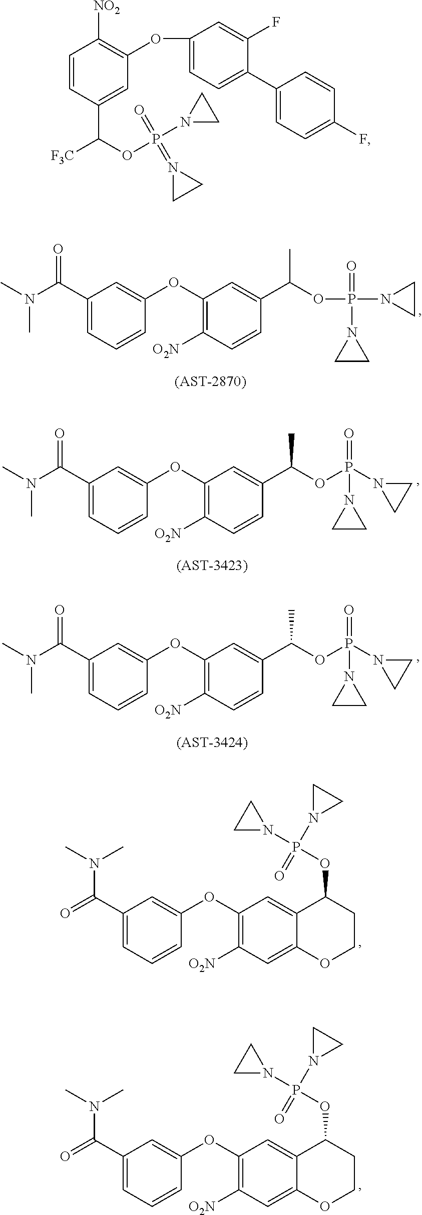

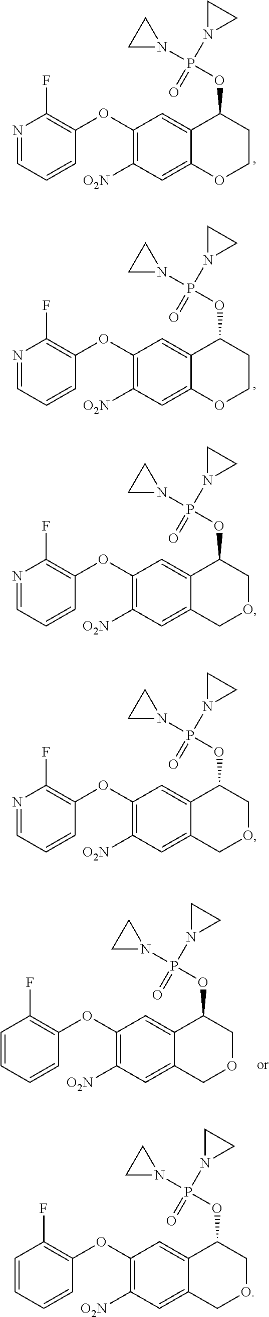

As a preference, according to the experimental results in the examples, a drug containing an AKR1C3-activated anticancer prodrug compound or its salt, ester, solvate or isotope isomer, is administered with a non-steroidal analgesic drug at an interval or simultaneously.

In the examples, experiments confirmed that some non-steroidal analgesics reported as inhibitors of AKR1C3 enzymes in some literatures significantly affect the anti-tumor cell proliferation activity of AST-3424 which is a drug activated by AKR1C3 enzymes; and some non-steroidal analgesics reported as inhibitors of AKR1C3 enzymes in some literatures do not affect the anti-tumor cell proliferation activity of AST-3424 which is a drug activated by AKR1C3 enzymes. The effect of the former non-steroidal analgesics on the anti-tumor cell proliferation activity of AST-3424 which is a drug activated by AKR1C3 enzyme can be eliminated after the cells were washed. Based on this, it is speculated that the effect will weaken or disappear after the drug exerting the effect is cleared or eliminated.

For nonsteroidal analgesics that do not affect the anti-tumor cell proliferation activity of the drug AST-3424 activated by AKR1C3 enzyme, it can be administered simultaneously with the AKR1C3 activated anticancer prodrug compound;
for nonsteroidal analgesics that affect the anti-tumor cell proliferation activity of the drug AST-3424 activated by AKR1C3 enzymes, it cannot be administered simultaneously with AKR1C3-activated anticancer prodrug compounds. AKR1C3-activated anticancer prodrug compounds should be administered at an interval after the nonsteroidal analgesics have been metabolized and cleared.

Simultaneous administration does not mean that they are administered strictly at the same time, but it actually means that the non-steroidal analgesic is administered during a period when the drug containing the AKR1C3 activated anticancer prodrug compound have not been completely metabolized, i.e., the AKR1C3 activated anticancer prodrug compound is still in the body, and the drug containing the AKR1C3 activated anticancer prodrug compound is administered during a period when the non-steroidal analgesic has not been completely metabolized, i.e., the non-steroidal analgesic is still in the body.

Administration at an interval includes two situations:
for the patient who has been administered with the non-steroidal analgesic drug, the drug containing the AKR1C3-activated anticancer prodrug compound or its salt, ester, solvate or isotope isomer will be administered only after the non-steroidal analgesic drug has been cleared; and
for the patient who has been administered with the drug containing the AKR1C3-activated anticancer prodrug compound or its salt, ester, solvate or isotope isomer, the non-steroidal analgesic drug will be administered only after the drug containing the AKR1C3-activated anticancer prodrug compound or its salt, ester, solvate or isotope isomer has been activated.

Preferably, the non-steroidal analgesic drug will be administered at a time interval of 1.33-1.75 hours after the start of administering the drug containing the AKR1C3-activated anticancer prodrug compound or its salt, ester, solvate or isotope isomer.

The compounds of structural formulae (1)-(2), similar to AST-3424, are the prodrugs of AST-2660. They can be activated by AKR1C3 enzyme (in the presence of NADPH) to generate AST-2660 (a DNA alkylating agent), so as to exert anti-cancer effect:

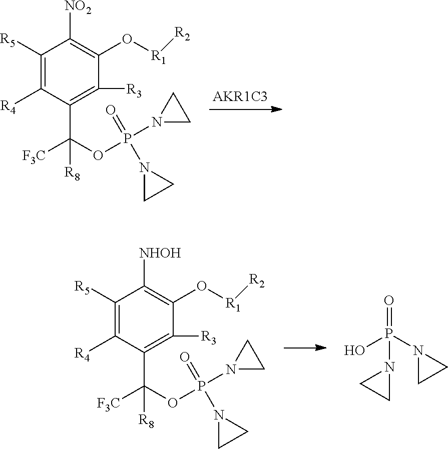

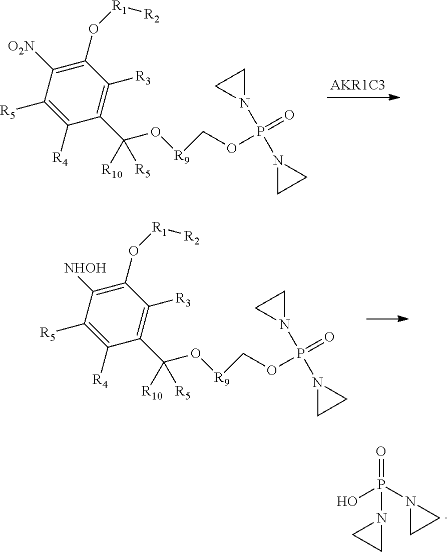

The compounds of structural formula (3) are the prodrugs of the nitrogen mustards

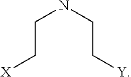

They can be activated by AKR1C3 enzyme (in the presence of NADPH) to generate the nitrogen mustards

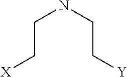

(a DNA alkylating agent), so as to exert anti-cancer effect:

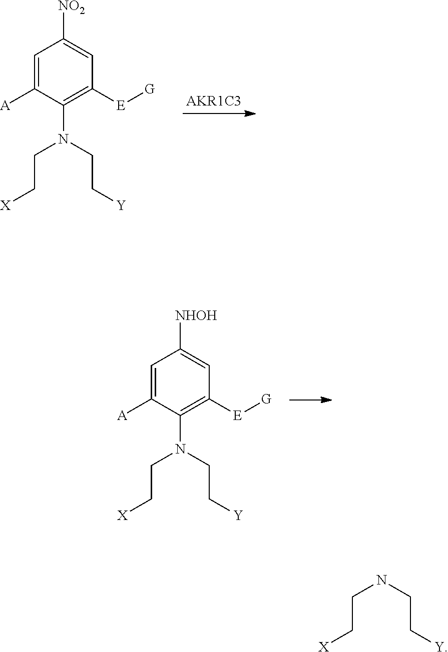

The compounds of structural formula (4), similar to AST-3424, are the prodrugs of AST-2660. They can be activated by AKR1C3 enzyme (in the presence of NADPH) to generate AST-2660 (a DNA alkylating agent), so as to exert anti-cancer effect:

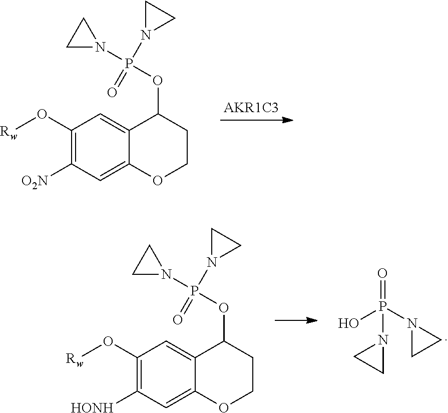

The compounds of structural formula (5), similar to AST-3424, are the prodrugs of phosphoramidate alkylating agent. They can be activated by AKR1C3 enzyme (in the presence of NADPH) to generate T (phosphoramidate alkylating agent, AST-2660 is a phosphoramidate alkylating agent), so as to exert anti-cancer effect:

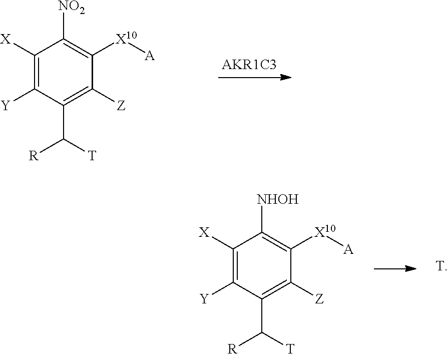

The compounds of structural formula (6), similar to AST-3424, are the prodrugs of AST-2660. They can be activated by AKR1C3 enzyme (in the presence of NADPH) to generate AST-2660, so as to exert anti-cancer effect:

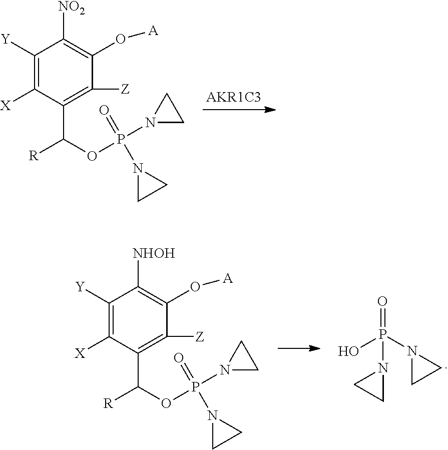

The compounds of structural formulas (7), (8) and (11), similar to AST-3424, can be activated by AKR1C3 enzyme (in the presence of NADPH) to generate drugs such as AST-2660, paclitaxel or camptothecin, so ad to exert anti-cancer effect.

The compounds of structural formulas (9)-(10) are the prodrugs of KARS inhibitor. They can be activated by AKR1C3 enzyme (in the presence of NADPH) to generate KARS inhibitor drugs, so as to exert anti-cancer effect.

According to the activation process of the above 11 types of AKR1C3-activated anticancer prodrug compounds, it can be seen that the activation of these compounds at the first stage requires the involvement of AKR1C3 enzyme, and after the activation is completed, even if non-steroidal analgesics have an inhibitory effect on AKR1C3 enzyme, the non-steroidal analgesics do not affect the above activation process (in other words, AKR1C3-activated anticancer prodrug compounds can still exert their efficacy).

Particularly, a drug containing AST-3424, AST-3423 or AST-2870 or its salt, ester, solvate or isotope isomer is administered simultaneously with Ibuprofen or Celecoxib.

According to the three-step analgesic ladder, non-steroidal analgesics are generally administered in the first step, which is aimed at mild pain. Therefore, the pain is mild pain.

The present invention actually provides a suitable dosing regimen for clinical cancer treatment. The effects of treating cancer and relieving pain are achieved simultaneously by combining AST-3424 with a non-steroidal analgesic drug.

The drug combination of the present invention comprises at least two active ingredients, such as the combination of AST-3424 and celecoxib, the combination of AST-3424 and naproxen, or the combination of AST-3424 and ibuprofen. Certainly, the drug combination may also comprise pharmaceutically acceptable excipients.

Typically, the two active ingredients that characterize the drug combination described herein may be formulated either together (single dosage unit) or separately (kit).

The two active ingredients, either formulated together or separately, may generally be administered simultaneously; or, given the properties of their respective preparation, the two active ingredients may be administered at an interval. The certain interval of time is conducive to optimizing the compatibility effect of the two active ingredients.

The drug combination can be a compound drug, which is a pharmaceutical composition consisting of the above-mentioned AKR1C3-activated anticancer prodrug compound or its salt, ester, solvate or isotope isomer as an active ingredient and the non-steroidal analgesic as another active ingredient. In this case, the above-mentioned active ingredients are formulated together and administered simultaneously.

The drug combination can also be a kit similar to Pfizer's COVID-19 drug Paxlovid, which can be sold together or separately. In this case, the above active ingredients are formulated separately, but two or more drugs which have been formulated separately can be administered simultaneously or separately with a certain interval of time according to the properties of their respective preparation.

In a specific embodiment, the active ingredients are formulated separately, wherein one drug of the formulated drugs contains active ingredients AST-3424, AST-3423, AST-2870 or its salts, esters, solvates or isotope isomers, and pharmaceutically acceptable excipients, and the other drug contains active ingredients Celecoxib, Naproxen or Ibuprofen and pharmaceutically acceptable excipients. The drug combination consisting of the above-mentioned two drugs can be administered simultaneously or separately with a certain interval of time according to the properties of their respective preparation.

In a particular embodiment, under the condition that the anti-tumor efficacy of AST-3424 are not affected or significantly affected, AST-3424 and celecoxib or ibuprofen can be administered simultaneously to relieve the patient's pain.

In a particular embodiment, under the condition that the anti-tumor efficacy of AST-3424 are not affected or significantly affected, AST-3424 and naproxen can be administered separately to relieve the patient's pain. AST-3424 can be administered first and then naproxen, or naproxen can be administered first and then AST-3424 can be administered after naproxen has been metabolized.

The present invention also provides a pharmaceutical formulation, which is prepared from the active ingredients of the above-mentioned drug combination and pharmaceutically acceptable excipients.

In a specific embodiment, the dosage form of the above-mentioned pharmaceutical formulation is selected from granules, tablets, pills, capsules, injections or lyophilized powder for injection.

The present invention also provides the use of the above-mentioned drug combination and/or the above-mentioned pharmaceutical formulation in the manufacture of anti-tumor drugs.

Optionally, the above cancer or tumor is selected from ovarian cancer, breast cancer, pancreatic cancer, fallopian tube cancer, primary peritoneal cancer, gastric cancer, prostate cancer, liver cancer, colon cancer, rectal cancer, lung cancer, and bladder cancer.

Preferably, the lung cancer is selected from non-small cell lung cancer and small cell lung cancer.

The present invention provides a method for treating cancer patients accompanied by pain by combining an AKR1C3-activated prodrug compound and an analgesic drug, a pharmaceutical use, a drug combination and a pharmaceutical formulation thereof, which provides a reasonable alternative for clinical cancer treatment. In particular, the combination of AST-3424 and a non-steroidal analgesic drug can achieve the effect of simultaneously treating cancer and relieving pain.

### BRIEF DESCRIPTION OF THE DRAWINGS

FIG. 1 shows the dose-effect curves of AST-3424 combination therapy with TH-3021, Celecoxib, Naproxen, and Ibuprofen on in vitro proliferation inhibition of H460 cell line, wherein Figure a involves TH-3021, Figure b involves Celecoxib, Figure c involves Naproxen, and Figure d involves Ibuprofen.

FIG. 2 shows the dose-effect curves of AST-3424 combination therapy with Naproxen on in vitro proliferation inhibition of H460 cell lines under different ways of combination therapy and different dosage, wherein Figure a shows the combined groups in which Naproxen or TH-3021 was added for two hours of pretreatment, and after the above two compounds were washed off, AST-3424 was added for two hours of treatment; Figure b shows the combined groups in which Naproxen or TH-3021 in combination with AST-3424 were added for two hours of co-treatment; Figure c shows the combined groups in which AST-3424 was added for two hours of pretreatment, and after the compound was washed off, Naproxen or TH-3021 was added for two hours of treatment; Figure d shows the combined groups in which Naproxen or TH-3021 was added for two hours of pretreatment, and without washing off the above two compounds, AST-3424 was added for two hours of co-treatment.

FIG. 3 shows the curve of changes in tumor volume over treatment time for each group of mice in the human lung cancer NCI-H460 model.

FIG. 4 shows the curve of changes in body weight over treatment time for each group of mice in the human lung cancer NCI-H460 model.

FIG. 5 shows the pharmacokinetic curves of OBI-3424 (A) and OBI-2660 (B) during cycle 1 (Days 1 and 8 combined) (extracted from FIG. 3 in the Phase I clinical trial poster of OBI-3424 issued by ASCO in 2022).

### EXAMPLES

### 1. In Vitro Cell Experiments

1.1 In Vitro Cell Proliferation Inhibition Experiment with the Drug Combination

The CTG detection method was used to evaluate the in vitro proliferation inhibitory effect of AST-3424 alone and in combination with Celecoxib, Naproxen, Ibuprofen, and TH-3021 respectively, on the H460 cell line.

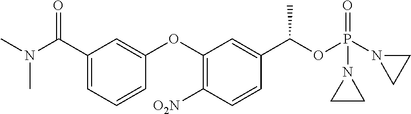 [SMILES: C[C@H](OP(=O)(N1CC1)N1CC1)c1ccc([N+](=O)[O-])c(Oc2cccc(C(=O)N(C)C)c2)c1]

(AST-3424) was synthesized with reference to the corresponding patent.

Celecoxib, Naproxen and Ibuprofen were all purchased from reagent companies.

According to research literature, TH-3021 is a specific inhibitor of AKR1C3 and can effectively inhibit the activation process of AKR1C3-activated anticancer prodrug compounds such as AST-3424 by AKR1C3, significantly weakening the ability of such compounds to inhibit the proliferation of tumor cells.

### 1.1.1 Cell Culture

a) H460 cells (human non-small lung cancer cells, AKR1C3 enzyme highly expressed) were cultured in RPMI-1640 medium, added with 10% FBS and 1% double antibody, and placed at 37° C. and 5% CO₂ for culturing.

### 1.1.2 Cell Seeding

a) The cells were routinely cultured until the cell saturation was 80%-90%, and when the required quantity was reached, the cells were collected.
b) The cells were resuspended with the corresponding medium and cell counts were performed to prepare a cell suspension with appropriate density.
c) The cell suspension was added to a 96-well plate with 100 μL per well, and the cell density was 2,000 cells per well.
d) The cells were incubated overnight in an incubator at 37° C. with 5% CO₂.
   1.1.3 Treating Cells with Compound (72 Hours)

AST-3424 alone: after 24 hours of cell seeding, AST-3424 was administered alone. Specifically, each well was supplemented with 99 μL of growth medium, and then added with 1 μL of the prepared AST-3424 solution to form different concentrations of AST-3424. The plate was shaken gently to ensure uniform mixing, and placed in an incubator at 37° C. with 5% CO₂.

The cell plates were placed in the incubator for 72 hours.

1.1.4 Treating Cells with Compound (2 Hours)
a) Administrating single drug: after 24 hours of cell seeding, AST-3424 was administered alone. Specifically, each well was supplemented with 99 μL of growth medium, and then added with 1 μL of the prepared AST-3424 solution to form different concentrations of AST-3424. The plate was shaken gently to ensure uniform mixing, and placed in the incubator for 2 hours. Then, the supernatant was discarded, and the remainder was washed twice with growth medium and added with 200 μL of fresh medium.
b) Administrating combined drugs: after 24 hours of cell seeding, AST-3424 was administered in combination with TH-3021, Celecoxib, Naproxen, and Ibuprofen respectively. Specifically, each well was supplemented with 98 μL of growth medium, and then added with 1 μL of the prepared TH-3021, Celecoxib, Naproxen, and Ibuprofen respectively, so as to form different concentrations of each compound. The plate was shaken gently to ensure uniform mixing. After the plate was placed in the incubator for 2 hours, 1 μL of the prepared AST-3424 solution was added to form different concentrations of AST-3424. The plate was shaken gently to ensure uniform mixing, and placed in the incubator at 37° C. with 5% CO₂ for another 2 hours. Then, the supernatant was discarded, and the remainder was washed twice with growth medium and added with 200 μL of fresh medium.

The cell plates were placed in the incubator for 72 hours.

### 1.1.5 Final Concentration of the Test Compounds:

### Concentration of Single Drug:

AST-3424 concentration: 100, 33.3, 11.1, 3.7, 1.23, 0.41, 0.14, 0.046, 0.015 [nM].

### Concentration of Drug Combination:

AST-3424 concentration: 100, 33.3, 11.1, 3.7, 1.23, 0.41, 0.14, 0.046, 0.015 [nM];
Celecoxib compound (2 #) 1 μM, 3 μM, 6 μM;
Naproxen compound (3 #) 200 μM, 300 μM, 400 μM;
Ibuprofen compound (4 #) 100 μM, 150 μM, 200 μM;
TH-3021 compound (5 #) 3 μM.

Referring to the pharmacokinetic parameters of celecoxib at single dosage (200 mg) in healthy subjects described in H20140107/H20140106 drug instructions from Pfizer or in drug instructions with National Medical Products Administration (NMPA) approval number H20193414 from Jiangsu Chiatai Qingjiang Pharmaceutical Co., Ltd., the Cmax of celecoxib is 705 ng/ml, which is converted to the molar concentration of 1.85 μmol/L. The concentration of the added celecoxib in the experiment was designed accordingly.

Referring to the drug instructions of U.S. FDA application number NDA017581 filed by ATNAHS PHARMA US, the Cmax of Naproxen for Naproxen tablets is 97.4 μg/ml, which is converted to the molar concentration of 423 μmol/L. The concentration of the added Naproxen in the experiment was designed accordingly.

Referring to main pharmacokinetic parameters of ibuprofen after a single dose of fasting administration, as described in the article written by Li Xiaobin, Yu Ming, Wang Nan et al., Pharmacokinetics and bioequivalence study of ibuprofen tablets in healthy Chinese subjects [J]. Chinese Journal of Clinical Pharmacology, 2020, 36(22): 3600-3604, DOI: 10.13699/j.cnki.1001-6821.2020.22.007, the Cmax of ibuprofen in the reference preparation was 1.94×10⁴ ng/ml, which was converted to the molar concentration of 94.05 μmol/L. The concentration of the added ibuprofen in the experiment was designed accordingly.

1.1.6 Detecting with CTG Method
a) The cell plate to be tested was placed at room temperature to equilibrium for 30 minutes, and 100 μL of culture medium was discarded from each well.
b) 25 μL CTG reagent (CelltiterGlo kit) was added to each well. The plate was placed on a fast shaker to shake for 2 minutes, and then placed at room temperature away from light for 30 minutes.
c) The chemiluminescent signal value was read with multifunctional microplate reader at reading time of 1000 ms.

### 1.1.7 Data Analysis

IC₅₀ was calculated with GraphPad Prism 5 software, and the IC50 (half maximal inhibitory concentration) of the compound was obtained using the following nonlinear fitting formula:

$$Y = Bottom + ( Top - Bottom ) / ( 1 + 10 \hat{} ( ( Log IC 50 - X ) * HillSlope ) ) ;$$

$$X : log value of compound concentration ,$$

$$Y : inhibition rate ( \% inhibition ) ;$$

$$Inhibition rate ( \% inhibition ) = 100 - ( ( reading of well containing compound - reading of low - read control ) / \\ ( reading of high - read control - reading of low - read control ) * 100 )$$

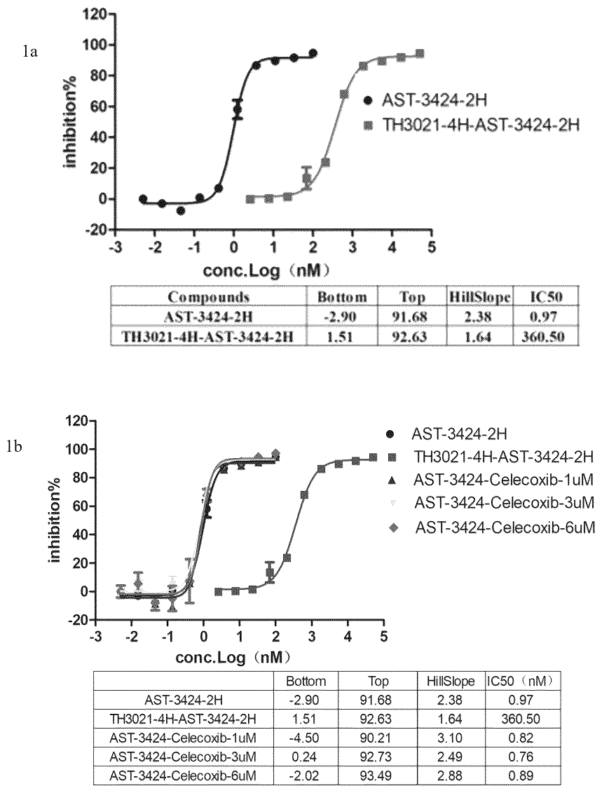

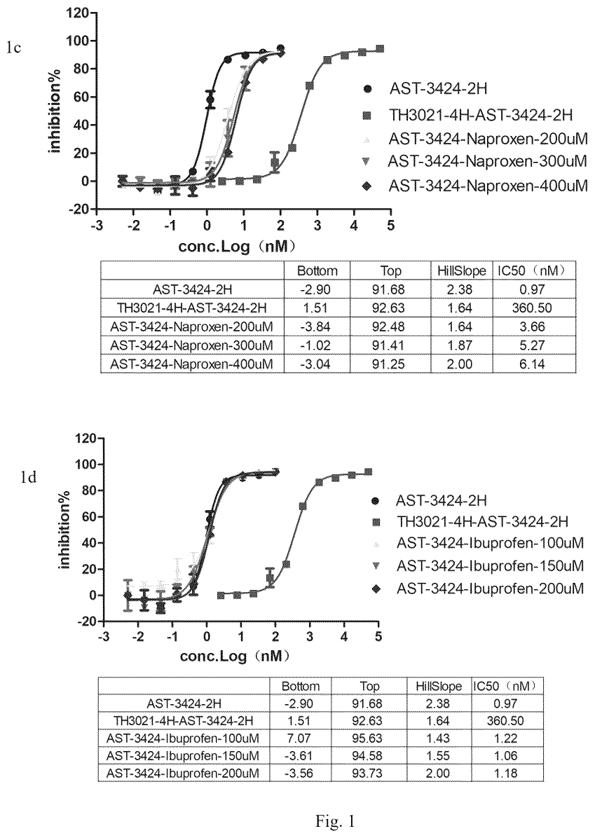

The dose-effect curve was obtained, as shown in FIG. 1, and the half maximal inhibitory concentration (IC₅₀) of the compound on cells was calculated, as shown in the following Table 1.

TABLE 1
In vitro proliferation inhibition data of H460 cell line
by the administration of AST-3424 alone or in combination
with Celecoxib, Naproxen, Ibuprofen, and TH-3021

| | IC₅₀(nM) | | |
|---|---|---|---|
| | | +3 μM | Inhibition |
| Test compound | -TH-3021 | TH-3021 | factor |
| AST-3424 | 0.97 | 360.50 | 371.65 |

| | | |
|---|---|---|
| AST-3424-Celecoxib-1 μM | 0.82 | 0.85 |
| AST-3424-Celecoxib-3 μM | 0.76 | 0.78 |
| AST-3424-Celecoxib-6 μM | 0.89 | 0.92 |
| AST-3424-Naproxen-200 μM | 3.66 | 3.77 |
| AST-3424-Naproxen-300 μM | 5.27 | 5.43 |
| AST-3424-Naproxen-400 μM | 6.14 | 6.33 |
| AST-3424-Ibuprofen-100 μM | 1.22 | 1.26 |
| AST-3424-Ibuprofen-150 μM | 1.06 | 1.09 |
| AST-3424-Ibuprofen-200 μM | 1.18 | 1.22 |

### Experimental Results

By comparison, it can be found that the combination of AST-3424 with celecoxib and ibuprofen respectively has almost no effect on the cell proliferation inhibitory activity of AST-3424 (AKR1C3-activated anticancer prodrug compound); however, the combination of AST-3424 with naproxen significantly affect the cell proliferation inhibitory activity of AST-3424, but the effect is far lower than that of the AKR1C3-specific inhibitor TH-3021 reported in the literature.

### 1.2 Experiment on Cell Proliferation Inhibition by Combining Naproxen and AST-3424 in Different Sequence of Administration

In order to further investigate the inhibitory mechanism of naproxen, a non-steroidal analgesic drug, which can significantly affect the cell proliferation inhibitory activity of AKR1C3-activated anticancer prodrug compounds such as AST-3424, experiments were performed with combined drugs in different sequence of administration.

The operations similar to those described in 1.1.1, 1.1.2, 1.1.6, and 1.1.7 above will not be described again, and the process of treating cells with the compounds will be directly described.

Treating Cells with the Compounds

a) Administrating the combined drugs:

After 24 hours of cell seeding, Naproxen and TH-3021 were respectively combined with AST-3424. The following are the different treatment methods.

1) TH-3021-3 M-2h-pretreatment-AST-3424-2h or Naproxen-400 μM-2h-pretreatment-AST-3424-2h: the combined groups include that after the pretreatment with Naproxen or TH-3021 for 2 hours, the above two compounds were washed off and then AST-3424 was added for 2 hours of treatment.

After 24 hours of cell seeding, Naproxen or TH-3021 was administered in combination with AST-3424.

Each well was supplemented with 99 μL of growth medium, and then added with 1 μL of the prepared Naproxen or TH-3021 solution. The plate was shaken gently to ensure uniform mixing, and placed in the incubator for 2 hours. Then the compounds were washed off, and each well was added with 99 μL of fresh medium, followed by 1 μL of the prepared AST-3424 solution to form different concentrations of AST-3424. The plate was shaken gently to ensure uniform mixing, and placed in the incubator for 2 hours. Subsequently, the plate was washed to remove the compound, and placed in the incubator at 37° C. with 5% CO₂ for further 72 hours.

2) TH-3021-3 μM+AST-3424-2h-simultaneously or Naproxen-400 μM+AST-3424-2h-simultaneously: the combined groups include that Naproxen or TH-3021 was administered in combination with AST-3424 for 2 hours of co-treatment.

After 24 hours of cell seeding, Naproxen or TH-3021 was administered in combination with AST-3424.

Each well was supplemented with 98 μL of growth medium, and then added with 1 μL of the prepared Naproxen or TH-3021 solution. The plate was shaken gently to ensure uniform mixing, and then each well was added with 1 μL of the prepared AST-3424 solution. The plate was shaken gently to ensure uniform mixing, and placed in the incubator for 2 hours. Subsequently, the plate was washed to remove the compounds, and placed in the incubator at 37° C. with 5% CO₂ for 72 hours.

3) AST-3424-2h-pretreatment-TH-3021-3 μM-2h or AST-3424-2h-pretreatment-Naproxen-400 μM-2h: the combined groups include that after the pretreatment with AST-3424 for 2 hours, the compound was washed off and then Naproxen or TH-3021 was respectively added for 2 hours of treatment.

After 24 hours of cell seeding, AST-3424 was administered in combination with Naproxen or TH-3021.

Each well was supplemented with 99 μL of growth medium and then added with 1 μL of the prepared AST-3424 solution. The plate was shaken gently to ensure uniform mixing, and placed in the incubator for 2 hours. Then the compound was washed off, and each well was added with 99 μL of fresh medium again, followed by 1 μL of the prepared Naproxen or TH-3021 solution. The plate was shaken gently to ensure uniform mixing, and placed in the incubator for 2 hours. Subsequently, the plate was washed to remove the compounds, and placed in the incubator at 37° C. with 5% CO₂ for further 72 hours.

4) TH-3021-3 μM-2h-pretreatment-AST-3424-2h-cotreatment or Naproxen-400 μM-2h-pretreatment-AST-3424-2h-cotreatment: the combined groups include after the pretreatment with Naproxen or TH-3021 for 2 hours, without washing off the above two compounds, AST-3424 was added for 2 hours of co-treatment.

After 24 hours of cell seeding, Naproxen or TH-3021 was administered in combination with AST-3424.

Each well was supplemented with 98 μL of growth medium, and then added with 1 μL of the prepared Naproxen or TH-3021 solution. The plate was shaken gently to ensure uniform mixing, and placed in the incubator for 2 hours. Then, each well was added with 1 μL of the prepared AST-3424 solution. The plate was shaken gently to ensure uniform mixing, and placed in the incubator for 2 hours. Subsequently, the plate was washed to remove the compounds, and placed in the incubator at 37° C. with 5% CO₂ for further 72 hours.

b) Administrating single drug: after 24 hours of cell seeding, the compound was administered alone. Specifically, each well was supplemented with 99 μL of growth medium, and then added with 1 μL of the prepared AST-3424 solution. The plate was shaken gently to ensure uniform mixing, and placed in the incubator at 37° C. with 5% CO₂ for 2 hours. Subsequently, the compound was washed off, and each well was added with fresh medium. The plate was placed in the incubator at 37° C. with 5% CO₂ for further 72 hours.

c) Final concentration of the test compounds:

### Concentration of Single Drug:

AST-3424 concentration:
i.100, 33.33, 11.11, 3.70, 1.23, 0.41, 0.14, 0.046, 0.015, 0 [nM].
ii.10, 3.33, 1.11, 0.37, 0.123, 0.041, 0.014, 0.0046, 0.0015, 0 [nM]

### Concentration of Combined Drugs:

AST-3424 concentration:
i.50000, 16666.67, 5555.56, 1851.85, 617.28, 205.76, 68.59, 22.86, 7.62 [nM];
ii. 10000, 2000, 400, 80, 16, 3.2, 0.64, 0.13, 0.03 [nM];
iii.100, 33.33, 11.11, 3.70, 1.23, 0.41, 0.14, 0.05, 0.02 [nM].
Naproxen concentration: 400 [M];
TH-3021 concentration: 3 [M].

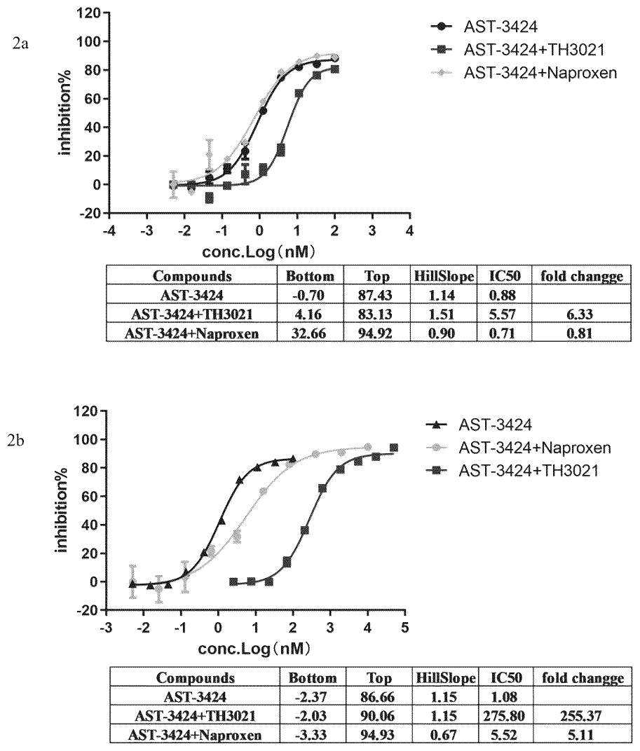

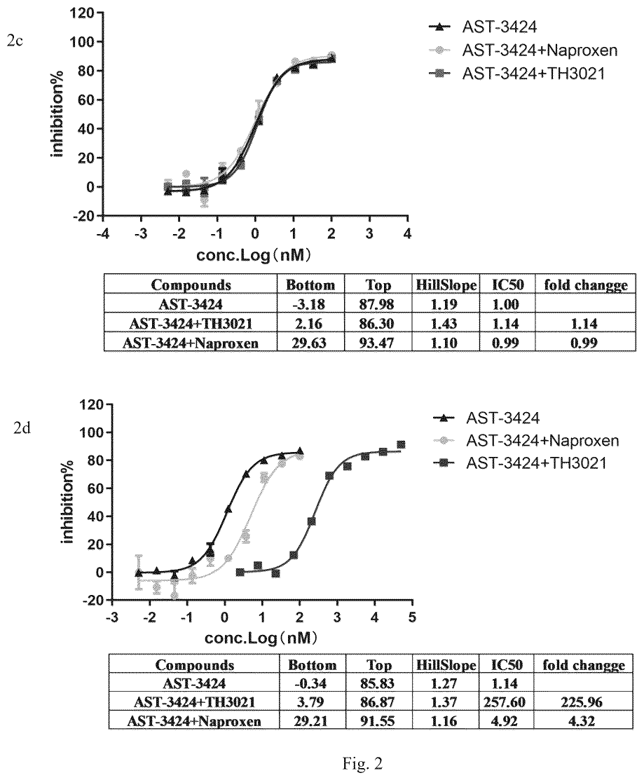

Finally, after data processing, the dose-effect curve was obtained, as shown in FIG. 2, and the half maximal inhibitory concentration (IC50) of AST-3424 on cells with different administration of combined drugs was calculated, as shown in Table 2 below.

TABLE 2
The half maximal inhibitory concentration (IC50) of AST-3424 on cells in the combination
experiment with different drugs and different sequence of administration

| Group | | | IC₅₀ | inhibition |
|---|---|---|---|---|
| number | The test compound | Treatment | (nM) | factor |
| 1 | AST-3424 | 2 h | 0.88 | / |
| | TH-3021 + AST-3424 | TH-3021-3 μM-2 h-pretreatment-AST-3424-2 h | 5.57 | 6.33 |
| | Naproxen + AST-3424 | Naproxen-400 μM-2 h-pretreatment-AST-3424-2 h | 0.71 | 0.81 |
| 2 | AST-3424 | 2 h | 1.08 | / |
| | AST-3424 + TH-3021 | TH-3021-3 μM AST-3424-2 h-simutaniously | 275.80 | 255.37 |
| | AST-3424 + Naproxen | Naproxen-400 μM + AST-3424-2 h-simutaniously | 5.52 | 5.11 |
| 3 | AST-3424 | 2 h | 1.00 | / |
| | AST-3424 + TH-3021 | AST-3424-2 h-pretreatment-TH-3021-3 μM-2 h | 1.14 | 1.14 |
| | AST-3424 + Naproxen | AST-3424-2 h-pretreatment-Naproxen-400 μM-2 h | 0.99 | 0.99 |
| 4 | AST-3424 | 2 h | 1.14 | / |
| | AST-3424 + TH-3021 | TH-3021-3 μM-2 h-pretreatment-AST-3424-2 h-cotreatment | 257.60 | 225.96 |
| | AST-3424 + Naproxen | Naproxen-400 μM-2 h-pretreatment-AST-3424-2 h- | 4.92 | 4.32 |
| | | cotreatment | | |

### Experimental Results

Analysis of the effect of TH-3021 on intracellular AKR1C3 enzyme activating AST-3424 showed that:

First, groups 1, 2, and 4 all demonstrated the effect of TH-3021 on the activation process of AST-3424. The addition of TH-3021 in advance for pretreating cells for 2 hours ensured that TH-3021 entered the cells and bound to the AKR1C3 enzyme. It was impossible for AST-3424 to be activated by the AKR1C3 enzyme to inhibit cell proliferation because the AKR1C3 enzyme had already bound to TH-3021 and could not activate AST-3424.

Second, groups 2 and 4 exhibited stronger inhibitory effect (200-fold difference in IC50 values), while group 1 showed relatively weaker inhibitory effect (6-fold difference). By comparing the cell treatment process of group 1 and those of groups 2 and 4, it can be seen that in group 1, the step of adding TH-3021 to the cell solution for 2 hours of incubation was followed by washing off the compound and adding culture medium again and AST-3424 drug, which resulted in the elution of TH-3021. This affected the inhibitory effect of TH-3021 on the enzyme activation process of AST-3424. Therefore, in group 1, although the effect of TH-3021 existed, it was relatively small.

Third, because AST-3424 had already been in contact with the cells and cultured for 2 hours in Group 3, the drug had already entered the cells and was fully activated by the AKR1C3 enzyme to generate AST-2660 that could inhibit cell proliferation. In this case, the subsequent addition of TH-3021 no longer affected the efficacy of AST-3424.

Analysis of the effect of Naproxen on intracellular AKR1C3 enzyme activating AST-3424 showed that:

First, groups 2 and 4 all demonstrated the effect of Naproxen on the activation process of AST-3424. In group 2, the treatment process included adding Naproxen and AST-3424 to the cell system simultaneously and culturing the cells for 2 hours, and in group 4, the treatment process included adding Naproxen to the cell system and culturing the cells for 2 hours before the addition of AST-3424. After contacting Naproxen with the cells and culturing for 2 hours, Naproxen had already bound to the AKR1C3 enzyme in the cells, thereby affecting the enzyme activation process of AST-3424 activated by AKR1C3 enzyme to generate AST-2660.

Second, in group 3, because AST-3424 had been in contact with cells and cultured for 2 hours, the drug had already entered the cells and was fully activated by the AKR1C3 enzyme to generate AST-2660 that could inhibit cell proliferation. In this case, the subsequent addition of Naproxen no longer affected the efficacy of AST-3424.

Third, due to the structure differences between Naproxen and TH-3021, Naproxen exhibited much lower ability than TH-3021 to enter cells, bind to the AKR1C3 enzyme, and affect the enzyme activation process of AST-3424 activated by the AKR1C3 enzyme to generate AST-2660. Therefore, incubating cells with Naproxen for 2 hours basically cannot achieve the pre-inhibition to the AKR1C3 enzyme, and the subsequent washing away of Naproxen directly led to the disappearance of its influence on the enzyme activation process of AST-3424. Thus, it was not showed in group 1 that Naproxen had an effect on the enzyme activation process of AST-3424 activated by AKR1C3 enzyme to generate AST-2660.

In summary, the conclusions are drawn as follows:

As compared with Naproxen, TH-3021 exhibited much greater inhibitory effect on the enzyme activation process of AST-3424 activated by AKR1C3 enzyme to generate AST-2660.

If AST-3424 was added first and in contact with cells for the incubation, the enzyme activation process of AST-3424 activated by AKR1C3 enzyme to generate AST-2660 occurred quickly; after a period of time (such as 2 hours shown in the experimental conditions), AST-3424 was converted completely to AST2660 and retained in the cells. The subsequent addition of Naproxen or TH-3021 no longer affected the results of cell proliferation inhibition.

If an AKR1C3 enzyme inhibitor (Naproxen or TH-3021) was added first and in contact with cells for a period of time (such as 2 hours shown in the experimental conditions), the inhibitor bound to the enzyme. However, this binding may be reversible to a large extent. After the inhibitor was washed off, whether the enzyme activation process of AST-3424 activated by AKR1C3 enzyme to generate AST-2660 would be significantly affected depended on the inhibition level of the inhibitor. Strong inhibitors such as TH-3021 can still show obvious inhibitory effect on the enzyme activation process, while weak inhibitors such as Naproxen cannot exhibit inhibitory effect on the enzyme activation process due to too short contact time with cells.

If AST-3424 and AKR1C3 enzyme inhibitor (Naproxen or TH-3021) were added simultaneously, the enzyme activation process of AST-3424 activated by AKR1C3 enzyme to generate AST-2660 was significantly affected.

Therefore, for the patient who has been administered with the non-steroidal analgesic drug that exhibits inhibitory effects on the enzyme activation process of AST-3424 activated by AKR1C3 enzyme to generate AST-2660, the drug containing the AKR1C3-activated anticancer prodrug compound or its salt, ester, solvate or isotope isomer will be administered only after the non-steroidal analgesic drug has been cleared. This administration manner of the drugs prevents the non-steroidal analgesic drug from inhibiting the enzyme activation process of AST-3424 activated by AKR1C3 enzyme to generate AST-2660.

For the patient who has been administered with the drug containing the AKR1C3-activated anticancer prodrug compound or its salt, ester, solvate or isotope isomer, the non-steroidal analgesic drug will be administered only after the AKR1C3-activated anticancer prodrug compound has been completely or substantially activated by AKR1C3 enzyme to generate AST-2660.

2. Pharmacodynamics and Safety Evaluations Experiment of Celecoxib Combined with AST-3424 in the Animal Model

The animal model experiment of celecoxib combination therapy with AST-3424 was performed, in order to further investigate the in vivo pharmacodynamics and safety of the combination therapy of non-steroidal analgesic drug celecoxib that does not significantly affect the cell proliferation inhibitory activity of AKR1C3-activated anticancer prodrug compounds such as AST-3424.

Measures of pharmacodynamics of AST-3424 monotherapy and combination therapy with Celecoxib were performed in a subcutaneous xenograft model of human lung cancer NCI-H460 established by using female BALB/c nude mice.

BALB/c nude mice were subcutaneously inoculated with human lung cancer NCI-H460 cells to establish the subcutaneous transplant tumor model of human lung cancer. The experiment was assigned to seven groups, with 5 mice in each group: the test drug Celecoxib 50 mg/kg and 100 mg/kg groups, the AST-3424 2.5 mg/kg group, and different combination therapy groups of Celecoxib 50 mg/kg or 100 mg/kg and AST-3424 2.5 mg/kg, and the 0.5% sodium carboxymethylcellulose vehicle control group. Among them, the 0.5% sodium carboxymethylcellulose vehicle control group, Celecoxib 50 mg/kg and 100 mg/kg groups were all administered by gavage once a day for a total of 15 days, and observed for 13 days. The AST-3424 2.5 mg/kg group was administered by tail vein injection once a week for a total of three weeks, and observed for 7 days. In the combination therapy groups of Celecoxib 50 mg/kg or 100 mg/kg and AST-3424 2.5 mg/kg, Celecoxib was administered by gavage once a day for a total of 15 days, and AST-3424 was administered by tail vein injection once a week for a total of three weeks, and observed for 7 days.

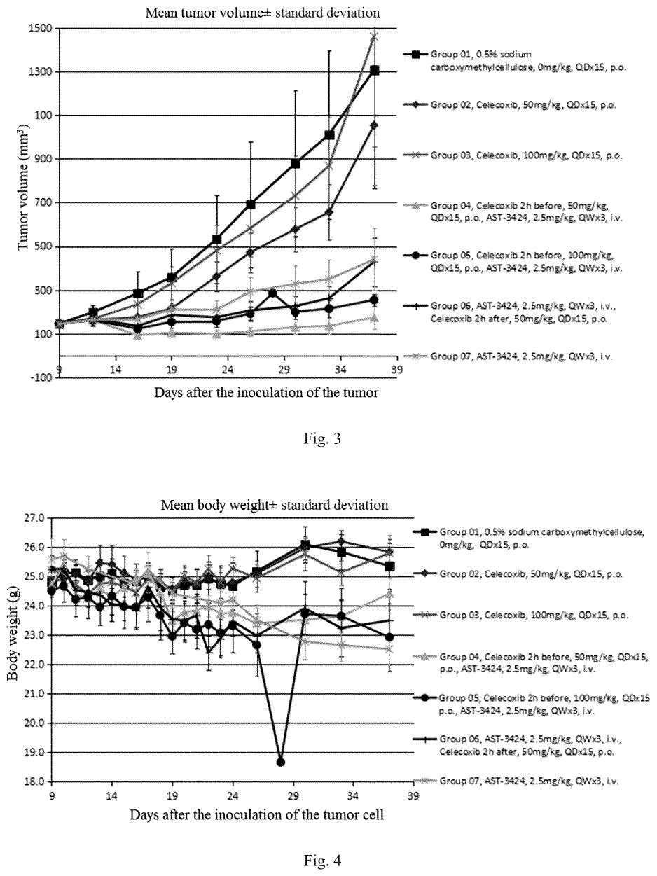

After the inoculation of tumor cell, tumor growth was routinely monitored, and tumor volume and mouse weight were measured. Evaluation of therapeutic efficiency was based on the relative tumor growth inhibition TGI (%). The specific results are shown in Table 3 and FIG. 3, and the Pharmacodynamics analysis data on Day 30 after inoculation (at the end of drug administration) are particularly given in Table 4.

The body weights of mice after inoculation were measured, and the results are shown in Table 5 and FIG. 4.

TABLE 3
Data table of changes in tumor volume of mice in each group over treatment time in the human lung cancer NCI-H460 model

| | Tumor volume (mm³) (x̅ ± S) | | | | | | |
|---|---|---|---|---|---|---|---|
| time | Group 1 | Group 2 | Group 3 | Group 4 | Group 5 | Group 6 | Group 7 |
| Day 9 after | 145.91 ± 15.40 | 145.68 ± 12.36 | 144.13 ± 11.94 | 145.66 ± 17.74 | 145.53 ± 10.50 | 145.75 ± 16.33 | 144.01 ± 11.36 |
| cell | | | | | | | |
| inoculation | | | | | | | |
| Day 12 after | 197.53 ± 35.10 | 162.55 ± 9.74 | 166.97 ± 17.80 | 161.77 ± 20.62 | 161.61 ± 18.28 | 168.74 ± 32.84 | 164.28 ± 17.97 |
| cell | | | | | | | |
| inoculation | | | | | | | |
| Day 16 after | 284.65 ± 101.62 | 175.22 ± 11.61 | 233.79 ± 48.62 | 92.65 ± 10.91 | 122.35 ± 18.76 | 135.00 ± 30.23 | 166.22 ± 18.81 |
| cell | | | | | | | |
| inoculation | | | | | | | |
| Day 19 after | 358.51 ± 128.32 | 214.83 ± 26.19 | 331.06 ± 73.94 | 104.34 ± 11.16 | 153.97 ± 28.71 | 185.34 ± 46.84 | 209.80 ± 41.43 |
| cell | | | | | | | |
| inoculation | | | | | | | |
| Day 23 after | 532.28 ± 200.64 | 361.15 ± 68.57 | 479.72 ± 117.21 | 99.20 ± 16.22 | 158.10 ± 26.39 | 174.97 ± 31.69 | 208.60 ± 43.27 |
| cell | | | | | | | |
| inoculation | | | | | | | |
| Day 26 after | 691.52 ± 287.97 | 471.39 ± 90.94 | 581.86 ± 130.92 | 110.73 ± 20.27 | 191.22 ± 31.55 | 205.85 ± 42.80 | 291.82 ± 67.06 |
| cell | | | | | | | |
| inoculation | | | | | | | |
| Day 30 after | 878.68 ± 335.77 | 577.49 ± 102.95 | 730.52 ± 183.64 | 129.96 ± 29.72 | 199.96 ± 34.40 | 225.90 ± 46.26 | 327.47 ± 85.10 |
| cell | | | | | | | |
| inoculation | | | | | | | |
| Day 33 after | 1010.57 ± 384.25 | 655.39 ± 126.55 | 867.83 ± 225.15 | 135.73 ± 34.82 | 214.28 ± 37.16 | 261.81 ± 55.85 | 348.83 ± 90.34 |
| cell | | | | | | | |
| inoculation | | | | | | | |
| Day 37 after | 1306.58 ± 540.88 | 1053.12 ± 274.37 | 1459.88 ± 505.50 | 173.92 ± 52.12 | 254.04 ± 27.55 | 428.29 ± 112.07 | 439.52 ± 142.78 |
| cell | | | | | | | |
| inoculation | | | | | | | |

TABLE 4
Pharmacodynamic analysis table of each group on Day30 after the
inoculation of tumor cell in the human lung cancer NCI-H460 model

| | On the 30th day after the inoculation of tumor cell (i.e. Day 30, | | | | |
|---|---|---|---|---|---|
| | on the 21st day after the initial administration of drugs, Nov. 9, 2020) | | | | |
| | | | | | P Value |
| | Tumor | Relative | | | (compared |
| Experimental | volume | tumor volume | TGI | T/C | to control |
| groups | (x̅ ± S) | (x̅ ± S) | (%) | (%) | group) |
| Group 1 | 878.68 ± 335.77 | 5.58 ± 1.64 | — | — | — |
| Group 2 | 577.49 ± 102.95 | 3.91 ± 0.45 | 29.87% | 70.13% | 1 |
| Group 3 | 730.52 ± 183.64 | 4.86 ± 0.82 | 12.84% | 87.16% | 9.84 × 10⁻¹ |
| Group 4 | 129.96 ± 29.72 | 0.96 ± 0.22 | 82.72% | 17.28% | 2.58 × 10⁻⁵ |
| Group 5 | 199.96 ± 34.40 | 1.48 ± 0.31 | 73.43% | 26.57% | 1.14 × 10⁻³ |
| Group 6 | 225.90 ± 46.26 | 1.51 ± 0.15 | 72.90% | 27.10% | 1.82 × 10⁻³ |
| Group 7 | 327.47 ± 85.10 | 2.19 ± 0.41 | 60.75% | 39.25% | 5.43 × 10⁻² |

TABLE 5
Body weight of mice of each group in the human lung cancer NCI-H460 model

| | The number of | | | | |
|---|---|---|---|---|---|
| | animals when | | | Change of body | |
| | Experiment | Mean body weight g (x̅ ± S) | | weight (%) | |
| | starts/ | Day 9 after the | Day 37 after the | Day 37 after the | The end |
| Experimental | experiment | inoculation of | inoculation of | inoculation of | point of |
| groups | ends | tumorcell | tumor cell | tumor cell | experiment |
| Group 1 | 5/5 | 24.8 ± 0.5 | 25.4 ± 0.8 | 2.20% | Day7 after the |
| Group 2 | 5/5 | 24.8 ± 0.5 | 25.8 ± 0.4 | 4.05% | end of all |
| Group 3 | 5/5 | 25.2 ± 0.5 | 25.8 ± 0.6 | 2.28% | administration |
| Group 4 | 5/5 | 24.7 ± 0.3 | 24.4 ± 0.5 | −1.05% | cycles (i.e. |
| Group 5 | 5/4 | 24.5 ± 0.5 | 22.9 ± 1.2 | −7.51% | Day37) |
| Group 6 | 5/5 | 25.3 ± 0.5 | 23.5 ± 1.0 | −7.11% | |
| Group 7 | 5/5 | 25.6 ± 0.7 | 22.5 ± 0.5 | −11.74% | |

Group 1, 0.5% sodium carboxymethylcellulose, 0 mg/kg, QD × 15, p.o.
Group 2, Celecoxib, 50 mg/kg, QD × 15, p.o.
Group 3, Celecoxib, 100 mg/kg, QD × 15, p.o.
Group 4, Celecoxib 2 h before, 50 mg/kg, QD × 15, p.o., AST-3424, 2.5 mg/kg, QW × 3, i.v.
Group 5, Celecoxib 2 h before, 100 mg/kg, QD × 15, p.o., AST-3424, 2.5 mg/kg, QW × 3, i.v.
Group 6, AST-3424, 2.5 mg/kg, QWx3, i.v., Celecoxib 2 h after, 50 mg/kg, QD × 15, p.o.
Group 7, AST-3424, 2.5 mg/kg, QWx3, i.v.

### Experimental Results

By comparison, it can be found that the TGI of AST-3424 monotherapy was 60.75%, and after combination therapy with celecoxib, the TGI did not decrease regardless of whether celecoxib was given before or after administrating AST-3424. Furthermore, compared to the AST-3424 monotherapy group, combination therapy group showed no significant difference in the tumor inhibition rate. In other words, the combination of the two drugs did not affect the tumor proliferation inhibition effect of AST-3424. Mice had good tolerance to both the AST-3424 monotherapy and combination therapy.

III. Pharmacokinetic Data Obtained from Phase I Clinical Trial of AST-3424

According to the pharmacokinetic data of OBI-3424 in the human clinical trial in the United States which is disclosed in the Phase I clinical trial poster of OBI-3424 issued by ASCO in 2022 (Safety, pharmacokinetics, and clinical activity of OBI-3424, an AKR1C3-activated prodrug, in patients with advanced or metastatic solid tumors: A phase 1 dose-escalation study., Apostolia Maria Tsimberidou, Claire F. Verschraegen, Pei Hsu, and Tillman E. Pearce, Journal of Clinical Oncology, 2022 40:16_suppl, 3030-3030, downloaded by the applicant from OBI Pharma Inc website, download URL: https://www.obipharma.com/zh-hant/news-zh-hant/poster-presentations-at-the-2022-asco-annual-meeting-for-adagloxad-simolenin-obi-999-and-obi-3424/, download date: Oct. 10, 2022), or referring to the relevant research paper published in May 2023 (Tsimberidou, A. M., Verschraegen, C. F., Wesolowski, R. et al. Phase 1 dose-escalation study evaluating the safety, pharmacokinetics, and clinical activity of OBI-3424 in patients with advanced or metastatic solid tumors. Br J Cancer 129, 266-274 (2023). https://doi.org/10.1038/s41416-023-02280-4), the following contents are described:

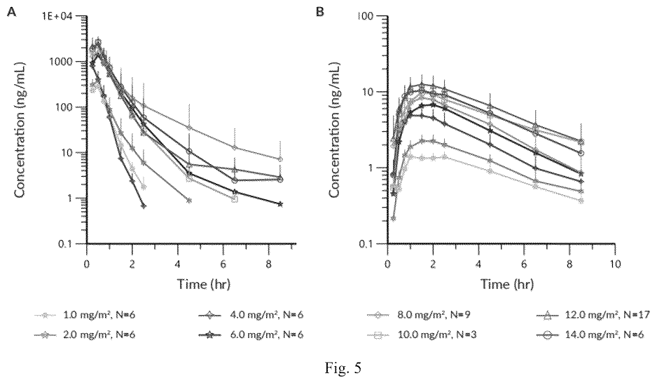

OBI-3424 and OBI-2660 concentrations were analyzed in blood samples collected on Day 1 of Cycle 1 (pretreatment); 15 minutes after the start of infusion; at the end of infusion (EOI); 15, 30, 60 and 90 minutes, and 2, 4, 6 and 8 hours after treatment. The curves of mean plasma concentration of a single dose of OBI-3424 and OBI-2660 versus time are shown in FIG. 5, and the pharmacokinetic parameters of OBI-3424 and OBI-2660 are summarized in Table 6.

Maximum serum concentrations (Cmax) of OBI-3424 generally occurred at the end of 30 minutes of drug infusion; Compared with OBI-3424, OBI-2660 had a slight delay in reaching maximum concentrations, with Cmax being achieved between 1.33 and 1.75 hours after the start of drug infusion.

OBI-3424 has a shorter half-life (from 0.20 to 0.74 hours), while OBI-2660 has a longer half-life (from 1.87 to 3.48 hours).

OBI-3424 has a mean clearance rate ranging from 4.8 to 8.8 L/h/m², and a volume of distribution ranging from 2.4 to 4.3 L/m².

For OBI-3424 and OBI-2660, no accumulation of exposure (Cmax and area under the concentration-time curve) was observed between the two doses (Cycle 1 Day1 and Cycle 1 Day8).

TABLE 6
Pharmacokinetic parameters of OBI-3424 and OBI-2660 (extracted from Table
3 in the Phase I clinical trial poster of OBI-3424 issued by ASCO in 2022
or Table 1 in the relevant research paper published in May 2023)

| Dose | Tₘₐₓ | | | | Cₘₐₓ | | | | AUC₀₋ₜ | |
|---|---|---|---|---|---|---|---|---|---|---|
| Level | (h) | | | | (ng/mL) | | | | (h*ng/mL) | |
| (mg/m²) | OBI-3424 | | OBI-2660 | | OBI-3424 | | OBI-2660 | | OBI-3424 | |
| 1 | 0.46 | (0.10) | 1.75 | (0.61) | 289.30 | (56.10) | 1.70 | (0.80) | 217.10 | (46.10) |
| 2 | 0.50 | 0.00 | 1.67 | (0.26) | 375.80 | (161.40) | 2.30 | (0.50) | 298.00 | (156.50) |
| 4 | 0.33 | (0.13) | 1.33 | (0.41) | 803.50 | (500.70) | 5.00 | (1.10) | 650.90 | (391.60) |
| 6 | 0.50 | 0.00 | 1.75 | (0.27) | 1360.30 | (247.10) | 6.80 | (0.40) | 1278.10 | (230.00) |
| 8 | 0.42 | (0.13) | 1.44 | (0.30) | 1613.10 | (419.80) | 8.50 | (1.60) | 1678.40 | (1302.00) |
| 10 | 0.50 | 0.00 | 1.67 | (0.29) | 2654.70 | (426.40) | 10.10 | (1.10) | 2037.00 | (316.20) |
| 12 | 0.44 | (0.17) | 1.53 | (0.37) | 2481.90 | (1132.70) | 13.10 | (4.20) | 2159.40 | (1089.10) |
| 14 | 0.42 | (0.13) | 1.75 | (0.52) | 2619.20 | (878.00) | 11.20 | (2.70) | 2131.80 | (955.10) |

| Dose | AUC₀₋ₜ | | T_{1/2} | | | | CL | | Vdₛₛ | |
|---|---|---|---|---|---|---|---|---|---|---|
| Level | (h*ng/mL) | | (h) | | | | (L/h/m²) | | (L/m²) | |
| (mg/m²) | OBI-2660 | | OBI-3424 | | OBI-2660 | | OBI-3424 | | OBI-3424 | |
| 1 | 7.30 | (1.90) | 0.29 | (0.04) | 3.08 | (1.21) | 4.74 | (0.87) | 2.45 | (0.40) |
| 2 | 10.00 | (2.20) | 0.31 | (0.14) | 2.48 | (0.76) | 8.85 | (5.25) | 4.13 | (1.67) |
| 4 | 19.30 | (6.70) | 0.21 | (0.04) | 2.00 | (0.24) | 7.74 | (3.29) | 2.68 | (1.69) |
| 6 | 27.40 | (3.50) | 0.74 | (0.39) | 2.09 | (0.44) | 4.84 | (1.03) | 3.72 | (0.83) |
| 8 | 32.80 | (8.90) | 0.67 | (0.33) | 1.87 | (0.35) | 6.53 | (2.92) | 3.97 | (1.06) |
| 10 | 29.20 | (18.30) | 0.57 | (0.24) | 2.99 | (0.10) | 4.92 | (0.83) | 3.06 | (0.50) |
| 12 | 56.60 | (21.40) | 0.55 | (0.34) | 2.41 | (0.69) | 8.20 | (7.98) | 4.33 | (4.67) |
| 14 | 45.60 | (9.80) | 0.55 | (0.27) | 2.46 | (0.78) | 8.49 | (5.67) | 4.31 | (1.22) |

Data are expressed as mean (SD).
CL, clearance rate;
AUC₀₋ₜ, area under the concentration-time curve during the period of time from 0 to the last measurable concentration;
Cmax, time to reach maximum concentration;
SD, standard deviation;
T_{1/2}, half-life;
Tmax, time to reach maximum concentration;
Vdss, distribution volume at steady state.

The published Phase I clinical data show that OBI-2660 reaches its maximum concentration between 1.33 and 1.75 hours after intravenous injection. At this time, the corresponding OBI-3424 is considered to be completely activated by the AKR1C3 enzyme and converted to OBI-2660. It is speculated that non-steroidal analgesics can be administered at this time.

1. A method for treating cancer patients accompanied by pain by combining a drug containing an AKR1C3-activated anticancer prodrug compound or its salt, ester, solvate or isotope isomer with a non-steroidal analgesic drug.

2. The method according to claim 1, wherein the AKR1C3-activated anticancer prodrug compound is selected from the compounds having structural formulae (1)-(11): 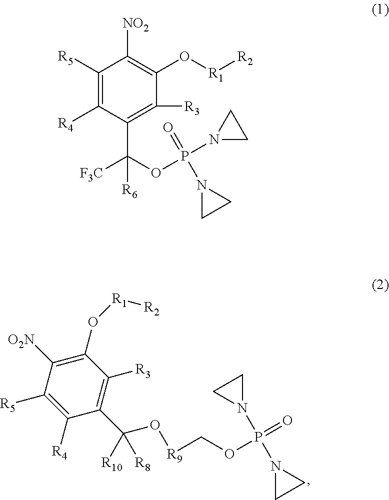
wherein, the definitions of R₁, R₂, R₃, R₄, R₅, R₈, R₉, and R₁₀ are described in the claims of Patent Application PCT/CN2020/089692 with Publication No. WO2020228685A1 (corresponding to Chinese Patent Application No. 2020800358890 with Publication No. CN113853379A); 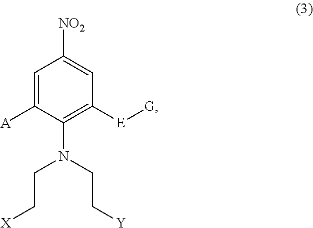
wherein, the definitions of A, E, G, X and Y are described in the claims of Patent Application PCT/NZ2019/050030 with Publication No. WO2019190331A1 (corresponding to Chinese Patent Application No. 2019800234236 with Publication No. CN111918864A); 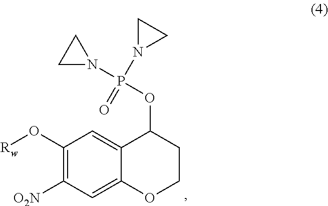
wherein, the definition of Rw is described in the claims of Patent Application PCT/CN2020/120281 with Publication No. WO2021068952A1 (corresponding to Chinese Patent Application No. 202080071652.8 with Publication No. CN 114555574A); 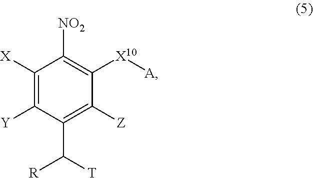
wherein, the definitions of X, Y, Z, R, T, A and X¹⁰ are described in the claims of Patent Application PCT/US2016/021581 with Publication No. WO2016145092A1 (corresponding to Chinese Patent Application No. 2016800150788 with Publication No. CN107530556A); 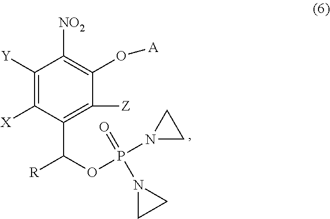
wherein:
A is a substituted or unsubstituted C₆-C₁₀ aryl, a biaryl or a substituted biaryl, a 5-15-membered heteroaryl, or —N═CR¹R²; wherein the substituent is selected from the group consisting of a halogen group, —CN, —NO₂, —O—(CH₂)—O—, —CO₂H and salt thereof, —OR¹⁰⁰, —CO₂R¹⁰⁰, —CONR¹⁰¹R¹⁰², —NR¹⁰¹R¹⁰², —NR¹⁰⁰SO₂R¹⁰⁰, —SO₂R¹⁰⁰, —SO₂NR¹⁰¹R¹⁰², C₁-C₆ alkyl, and C₃-C₁₀ heterocyclyl;
wherein R¹⁰⁰, R¹⁰¹ and R¹⁰² are each independently hydrogen, C₁-C₅ alkyl, or C₆-C₁₂ aryl; or R¹⁰¹ and R¹⁰² together with the nitrogen atom to which they are attached form a 5-7-membered heterocycle;
wherein the alkyl group and the aryl group are each substituted by 1-3 halogen groups or 1-3 C₁-C₆ alkyl groups;
R¹ and R² are each independently phenyl or methyl;
X, Y and Z are each independently hydrogen or a halogen group;
R is hydrogen or C₁-C₆ alkyl or halogen-substituted alkyl; 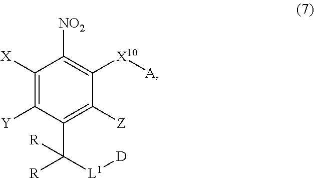
wherein, the definitions of X, Y, Z, R, D, L¹, A and X¹⁰ are described in the claims of Patent Application PCT/US2016/025665 with Publication No. WO2016161342A3 (corresponding to Chinese Patent Application No. 2016800200132 with Publication No. CN108136214A); 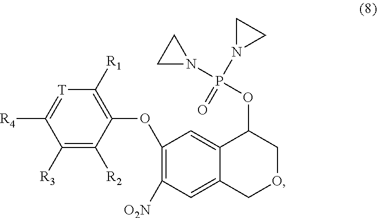
wherein, the definitions of R₁, R₂, R₃, R₄, and T are described in the claims of Patent Application PCT/CN2021/118597 with Publication No. WO2022057838A1; 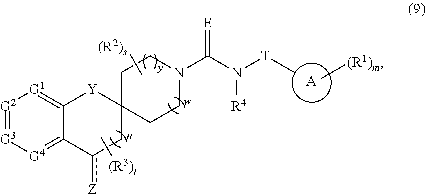
wherein, the definitions of R¹, R², R³, R⁴, G¹, G², G³, G⁴, E, T, Y, Z, m, n, s, t, v, w, and Ring A are described in the claims of Patent Application CN202210585771.6 with Publication No. CN115403579A; 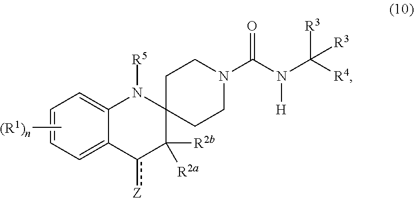
or a pharmaceutically acceptable salt thereof,
wherein, the definitions of R¹, R^{2a}, R^{2b}, R³, R⁴, R⁵, n, and Z are described in the claims of Patent Application PCT/IB2020/057285 with Publication No. WO2021005586A1 (corresponding to Chinese Patent Application No. CN202080053804.1 with Publication No. CN114206870A); 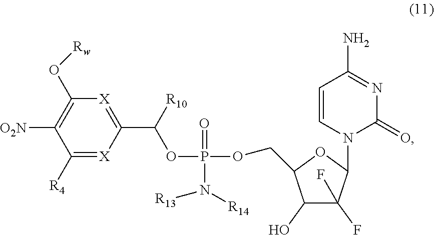
or a pharmaceutically acceptable salt thereof,
wherein, the definitions of R_{w}, X, R₄, R₁₀, R₁₃, and R₁₄ are described in the claims of Patent Application PCT/CN2022/098082 with Publication No. WO2022258043A1.

3. The method according to claim 1, wherein the AKR1C3-activated anticancer prodrug compound is selected from the compounds having the following structural formulae:

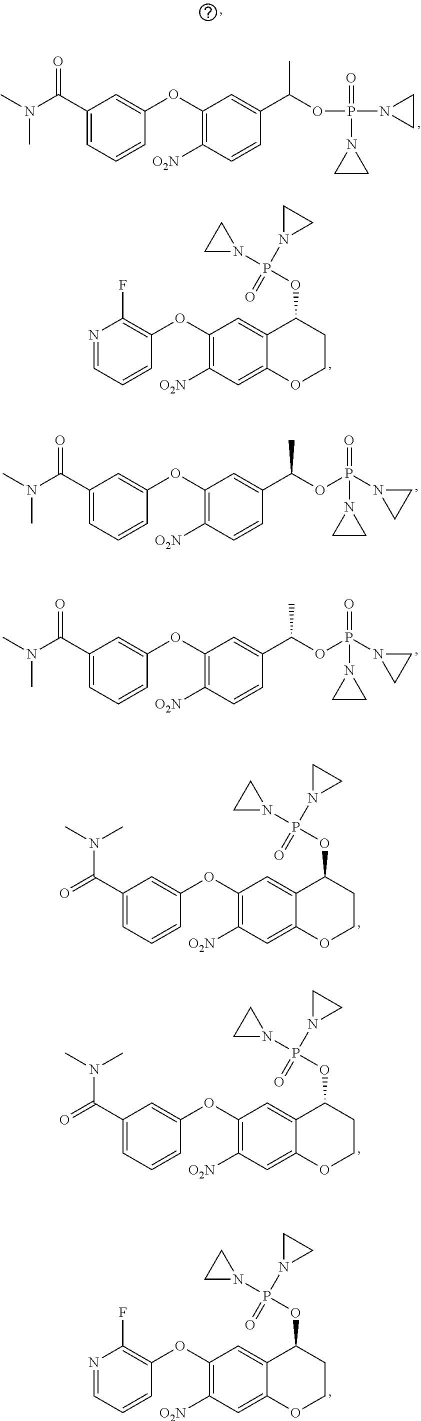

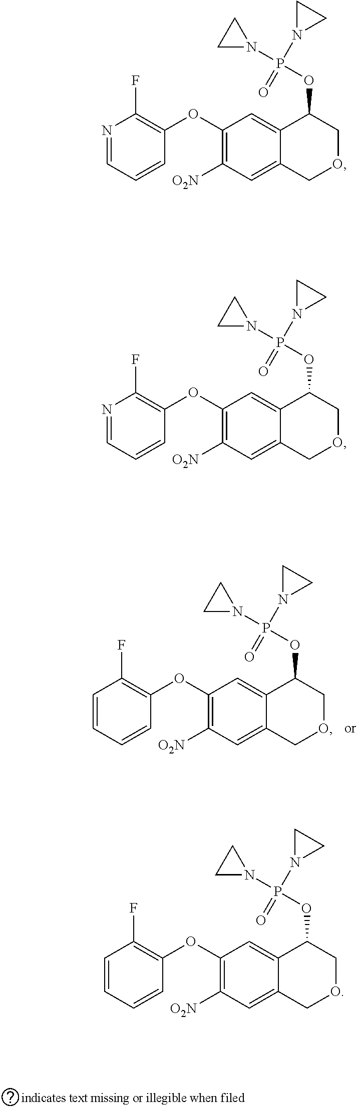 [SMILES: O=[N+]([O-])c1cc2c(cc1Oc1cccnc1F)[C@@H](OP(=O)(N1CC1)N1CC1)COC2 ; O=[N+]([O-])c1cc2c(cc1Oc1cccnc1F)[C@H](OP(=O)(N1CC1)N1CC1)COC2 ; O=[N+]([O-])c1cc2c(cc1Oc1ccccc1F)[C@@H](OP(=O)(N1CC1)N1CC1)COC2 ; O=[N+]([O-])c1cc2c(cc1Oc1ccccc1F)[C@H](OP(=O)(N1CC1)N1CC1)COC2]

4. The method according to claim 1, wherein the drug containing the AKR1C3-activated anticancer prodrug compound or its salt, ester, solvate or isotope isomer is administered with the non-steroidal analgesic drug at an interval or simultaneously.

5. The method according to claim 4, wherein, for the patient who has been administered with the non-steroidal analgesic drug, the drug containing the AKR1C3-activated anticancer prodrug compound or its salt, ester, solvate or isotope isomer will be administered only after the non-steroidal analgesic drug has been cleared; and
for the patient who has been administered with the drug containing the AKR1C3-activated anticancer prodrug compound or its salt, ester, solvate or isotope isomer, the non-steroidal analgesic drug will be administered only after the drug containing the AKR1C3-activated anticancer prodrug compound or its salt, ester, solvate or isotope isomer has been activated.

6. The method according to claim 5, wherein, the non-steroidal analgesic drug will be administered at a time interval of 1.33-1.75 hours after the start of administering the drug containing the AKR1C3-activated anticancer prodrug compound or its salt, ester, solvate or isotope isomer.

7. The method according to claim 4, wherein a drug containing AST-3424, AST-3423 or AST-2870 or its salt, ester, solvate or isotope isomer is administered simultaneously with Ibuprofen or Celecoxib.

8. The method according to claim 1, wherein the non-steroidal analgesic drug is selected from the group consisting of Aspirin, Sodium salicylate, Paracetamol, Indomethacin, Naproxen, Nabumetone, Diclofenac, Ibuprofen, rofecoxib, Celecoxib, Nimesulide, Aceclofenac, Diflunisal, Etodolac, Fenoprofen, Flurbiprofen, ketoprofen, suprofen, Tiaprofenic Acid, Ketorolac, Zomepirac, Mefenamic acid, Flufenamic acid, Meclofenamic Acid, Meloxicam, Oxaprozin, Piroxicam, Tenoxicam, Lornoxicam, Sasapyrine, Sulindac, Tolmetin, Phenacetin, Loxoprofen Sodium, Aminopyrine, Metamizole Sodium, Sudoxicam, Phenylbutazone, Oxyphenbutazone.

9. The method according to claim 1, wherein the pain is mild pain.

10.-11. (canceled)

12. A drug combination, characterized in that the following substances are comprised as active ingredients:
an AKR1C3-activated prodrug compound or its salt, ester, solvate or isotope isomer, and a non-steroidal analgesic drug;
wherein, the active ingredients are formulated together or separately for combined use, simultaneous use or separate use.

13. The drug combination according to claim 12, which is used to treat cancer or tumor patients accompanied by pain.

14. The drug combination according to claim 12, wherein the AKR1C3-activated anticancer prodrug compound is selected from the compounds having structural formulae (1)-(11): 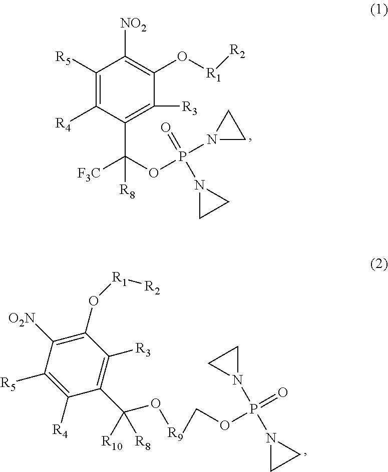
wherein, the definitions of R₁, R₂, R₃, R₄, R₅, R₈, R₉, and R₁₀ are described in the claims of Patent Application PCT/CN2020/089692 with Publication No. WO2020228685A1 (corresponding to Chinese Patent Application No. 2020800358890 with Publication No. CN113853379A); 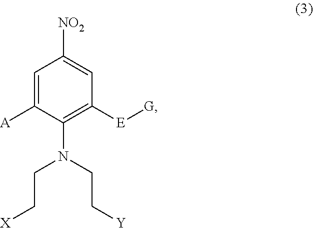
wherein, the definitions of A, E, G, X and Y are described in the claims of Patent Application PCT/NZ2019/050030 with Publication No. WO2019190331A1 (corresponding to Chinese Patent Application No. 2019800234236 with Publication No. CN 111918864A); 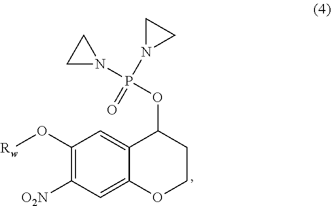
wherein, the definition of Rw is described in the claims of Patent Application PCT/CN2020/120281 with Publication No. WO2021068952A1 (corresponding to Chinese Patent Application No. 202080071652.8 with Publication No. CN114555574A); 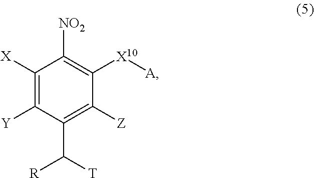
wherein, the definitions of X, Y, Z, R, T, A and X¹⁰ are described in the claims of Patent Application PCT/US2016/021581 with Publication No. WO2016145092A1 (corresponding to Chinese Patent Application No. 2016800150788 with Publication No. CN107530556A); 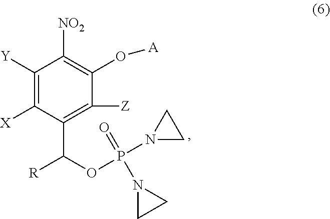
wherein:
A is a substituted or unsubstituted C₆-C₁₀ aryl, a biaryl or a substituted biaryl, a 5-15-membered heteroaryl, or —N═CR¹R²; wherein the substituent is selected from the group consisting of a halogen group, —CN, —NO₂, —O—(CH₂)—O—, —CO₂H and salt thereof, —OR¹⁰⁰, —CO₂R¹⁰⁰, —CONR¹⁰¹R¹⁰², —NR¹⁰¹R¹⁰², —NR¹⁰⁰SO₂R¹⁰⁰, —SO₂R¹⁰⁰, —SO₂NR¹⁰¹R¹⁰², C₁-C₆ alkyl, and C₃-C₁₀ heterocyclyl;
wherein R¹⁰⁰, R¹⁰¹ and R¹⁰² are each independently hydrogen, C₁-C₈ alkyl, or C₆-C₁₂ aryl; or R¹⁰¹ and R¹⁰² together with the nitrogen atom to which they are attached form a 5-7-membered heterocycle;
wherein the alkyl group and the aryl group are each substituted by 1-3 halogen groups or 1-3 C₁-C₆ alkyl groups;
R¹ and R² are each independently phenyl or methyl;
X, Y and Z are each independently hydrogen or a halogen group;
R is hydrogen or C₁-C₆ alkyl or halogen-substituted alkyl; 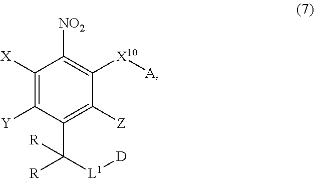
wherein, the definitions of X, Y, Z, R, D, L¹, A and X¹⁰ are described in the claims of Patent Application PCT/US2016/025665 with Publication No. WO2016161342A3 (corresponding to Chinese Patent Application No. 2016800200132 with Publication No. CN108136214A); 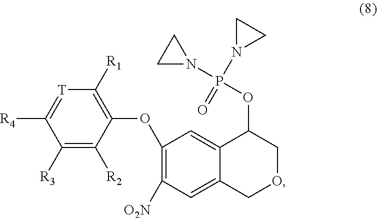
wherein, the definitions of R₁, R₂, R₃, R₄, and T are described in the claims of Patent Application PCT/CN2021/118597 with Publication No. WO2022057838A1; 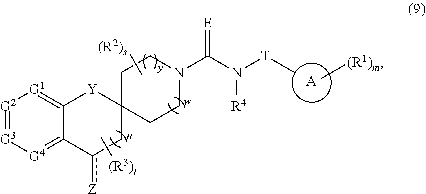
wherein, the definitions of R¹, R², R³, R⁴, G¹, G², G³, G⁴, E, T, Y, Z, m, n, s, t, v, w, and Ring A are described in the claims of Patent Application CN202210585771.6 with Publication No. CN115403579A; 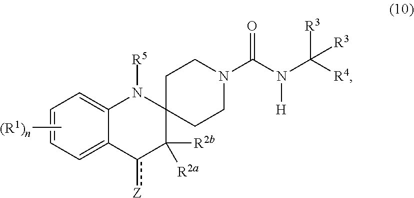
or a pharmaceutically acceptable salt thereof,
wherein, the definitions of R¹, R^{2a}, R^{2b}, R³, R⁴, R⁵, n, and Z are described in the claims of Patent Application PCT/IB2020/057285 with Publication No. WO2021005586A1 (corresponding to Chinese Patent Application No. CN202080053804.1 with Publication No. CN114206870A); 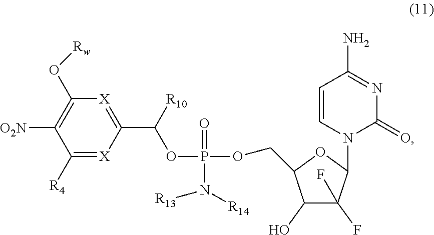
or a pharmaceutically acceptable salt thereof,
wherein, the definitions of R_{w}, X, R₄, R₁₀, R₁₃, and R₁₄ are described in the claims of Patent Application PCT/CN2022/098082 with Publication No. WO2022258043A1.

15. The drug combination according to claim 12, wherein the AKR1C3-activated anticancer prodrug is selected from the compounds having the following structural formulae:

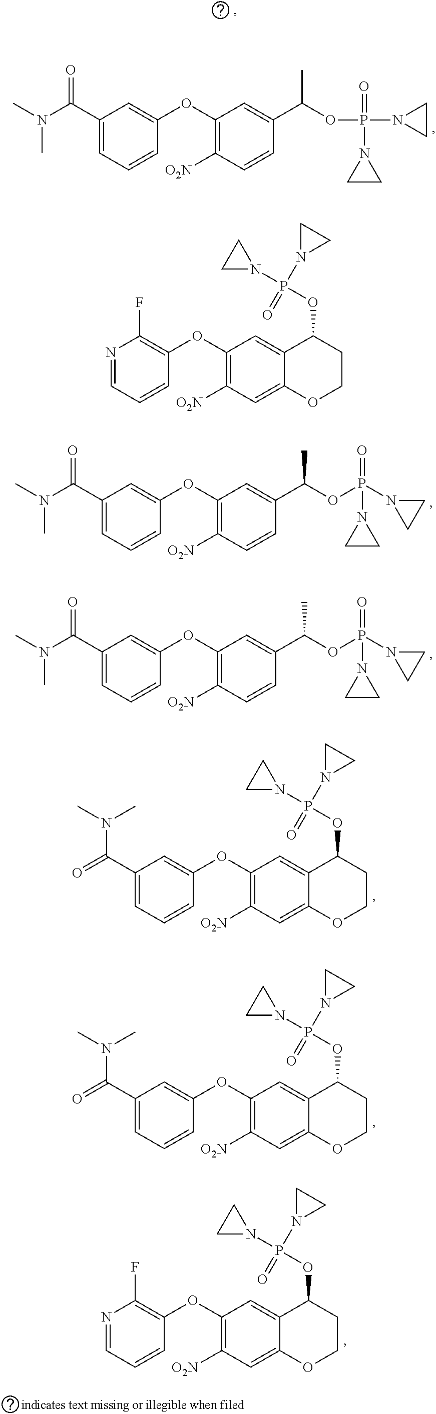

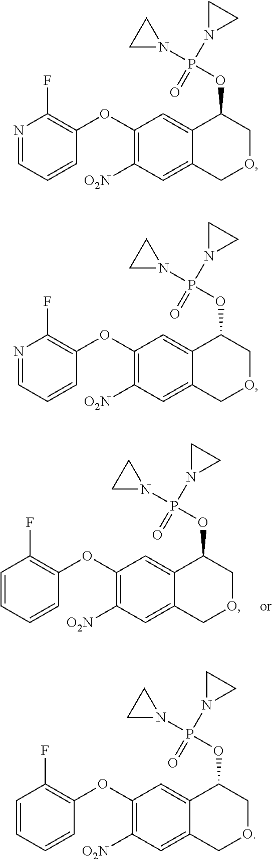

16. The drug combination according to claim 12, wherein the non-steroidal analgesic drug is selected from the group consisting of Aspirin, Sodium salicylate, Paracetamol, Indomethacin, Naproxen, Nabumetone, Diclofenac, Ibuprofen, rofecoxib, Celecoxib, Nimesulide, Aceclofenac, Diflunisal, Etodolac, Fenoprofen, Flurbiprofen, ketoprofen, suprofen, Tiaprofenic Acid, Ketorolac, Zomepirac, Mefenamic acid, Flufenamic acid, Meclofenamic Acid, Meloxicam, Oxaprozin, Piroxicam, Tenoxicam, Lornoxicam, Sasapyrine, Sulindac, Tolmetin, Phenacetin, Loxoprofen Sodium, Aminopyrine, Metamizole Sodium, Sudoxicam, Phenylbutazone, Oxyphenbutazone.

17. A pharmaceutical formulation, characterized in that the pharmaceutical formulation is prepared from the active ingredients of the drug combination according to claim 12 and pharmaceutically acceptable excipients.

18. The pharmaceutical formulation according to claim 17, characterized in that the dosage form of the pharmaceutical formulation is selected from the group consisting of granules, tablets, pills, capsules, injections, and lyophilized powder for injection.

19. A method for treating tumor, comprising administering the drug combination according to claim 12.

20. The drug combination according to claim 13, wherein the cancer or tumor is selected from the group consisting of ovarian cancer, breast cancer, pancreatic cancer, fallopian tube cancer, primary peritoneal cancer, gastric cancer, prostate cancer, liver cancer, colon cancer, rectal cancer, lung cancer, or bladder cancer.

21. The drug combination according to claim 20, wherein the lung cancer is non-cell lung cancer or small cell lung cancer.
